(19) United States
(12) Patent Application Publication
(10) Pub. No.: US 2023/0374607 A1
(43) Pub. Date: Nov. 23, 2023

(54) TRANSMEMBRANE NANOSENSOR ARRAYS FOR RAPID, ULTRA-SENSITIVE AND SPECIFIC DIGITAL QUANTIFICATION OF INTERNAL MICRO-RNA CONTENT OF INTACT EXOSOMES

(71) Applicant: ARIZONA BOARD OF REGENTS ON BEHALF OF ARIZONA STATE UNIVERSITY, Scottsdale, AZ (US)

(72) Inventors: Hao Yan, Gilbert, AZ (US); Rizal Hariadi, Phoenix, AZ (US); Swarup Dey, Middleton, WI (US)

(21) Appl. No.: 18/298,922

(22) Filed: Apr. 11, 2023

Related U.S. Application Data
(63) Continuation-in-part of application No. 17/799,755, filed on Aug. 15, 2022.
(60) Provisional application No. 62/977,454, filed on Feb. 17, 2020.

(51) Int. Cl. C12Q 1/6886; C12Q 1/6818; C12Q 1/6804

(52) U.S. Cl. CPC C12Q 1/6886; C12Q 1/6818; C12Q 2600/178; C12Q 1/6804

(57) ABSTRACT

Disclosed herein is a transmembrane nanosensor device comprising a lipid conjugated DNA tweezer, and methods of using the same.

### CROSS-REFERENCE TO RELATED APPLICATIONS

This application is a continuation-in-part application of U.S. Ser. No. 17/799,755, filed Aug. 15, 2022, which is the U.S. National Stage of PCT/US2021/018371 with international filing date Feb. 17, 2021, which claims the benefit of U.S. Application No. 62/977,454 filed on Feb. 17, 2020. The entire content of each of which of the above-referenced applications is incorporated herein by reference in its entirety.

### STATEMENT REGARDING FEDERALLY SPONSORED RESEARCH

This invention was made with government support under 1644745 awarded by the National Science Foundation. The government has certain rights in the invention.

### SEQUENCE LISTING

A sequence listing accompanies this application and is submitted as an ASCII file of the sequence listing named “112624_01398_sequences.xml” which is 59,525 bytes in size and was created on Jun. 12, 2023. The sequence listing is electronically submitted via EFS-Web with the application and is incorporated herein by reference in its entirety.

### FIELD

The field of the invention relates to a transmembrane nanosensor device comprising a lipid conjugated DNA tweezer, and methods of using the same.

### BACKGROUND

Surgical tumor tissue biopsy, considered as the gold standard for cancer diagnosis and prognosis, suffers from several problems such as patient inconvenience, slow rate, partial sampling and non-specific findings. To this end, non-invasive sampling of body fluids, known as liquid biopsy, offers great promise for cancer diagnosis and prognosis. Among several liquid biopsy biomarkers, exosomes, a type of extracellular vesicles (EVs), are particularly appealing due to their relative high abundance (8-500 thousand/μL), availability in several body fluids such as breast milk, urine, saliva etc. along with blood and enrichment with various biomarkers such as proteins and micro-RNA (miRNA) molecules. Current exosome-based diagnostic techniques rely on isolation of exosomes by tedious, expensive, and time-consuming methods that limit their translation to clinical set up. Moreover, many methods fail to distinguish tumor-derived exosomes from their healthy counterparts. Current methods of exosomal micro-RNA (ex-miRNA) profiling relies on isolation followed by lysis of the exosomes to extract miRNAs and then the extracted miRNAs are amplified and detected. This process requires special training, it is expensive, tedious and time consuming. Moreover, lysing mixes the ex-miRNA from different sub-populations of exosomes as well as with free-flowing miRNAs. This obscures the specificity and sensitivity of the detection. Improved exosomal biomarker profiling with high sensitivity, specificity and clinical feasibility will help translation of the ex-miRNA based liquid biopsy as a diagnostic and prognostic method.

Therefore, a need exists for improved exosomal biomarker specific diagnostic compositions and methods.

### SUMMARY

In a first aspect, provided herein is a transmembrane nanosensor device comprising a lipid conjugated DNA tweezer comprising a hairpin loop complementary to a target polynucleotide trigger strand; a fluorophore; and a quencher paired to the fluorophore, or a FRET pair, wherein when the hairpin loop is bound by the target polynucleotide trigger strand, the DNA tweezer transitions from a closed conformation to an open conformation, the quencher is separated from the fluorophore, and the fluorophore fluoresces. In some embodiments, the lipid conjugated DNA tweezer is integrated into a lipid bilayer. In some embodiments, the lipid bilayer is an exosome membrane. In some embodiments, the target polynucleotide trigger strand is an RNA or a DNA polynucleotide. In some embodiments, the target polynucleotide trigger strand is a micro RNA (miRNA). In some embodiments, the lipid is a cholesterol molecule. In some embodiments, the trigger strand is a disease biomarker, for example a cancer biomarker.

In a second aspect, provided herein is a method of diagnosing a disease in a subject comprising contacting exosomes from a subject with a transmembrane nanosensor as described herein; and measuring and quantifying fluorescence of the transmembrane nanosensor, wherein fluorescence of the nanosensor indicates the presence of the disease in the subject. In some embodiments, the exosomes are from a liquid biological sample from the subject. In some embodiments, the target polynucleotide trigger is an miRNA biomarker specific to cancer.

In a third aspect, provided herein is an exosomal nanoarray comprising exosomes bound to a solid surface and a transmembrane nanosensor as described herein. In some embodiments, the exosomes are bound to the solid surface by an exosome specific antibody. In some embodiments, the exosome specific antibody is anti-CD81. In some embodiments, the exosomes are from a patient sample. In some embodiments, the patient sample is a liquid biological sample.

In a fourth aspect, provided herein is a transmembrane nanosensor device. The device includes a lipid conjugated DNA tweezer comprising a hairpin loop complementary to a target polynucleotide trigger strand; a fluorophore; a quencher paired to the fluorophore, and an initiator sequence. When the hairpin loop is bound by the target polynucleotide trigger strand, the DNA tweezer transitions from a closed conformation to an open conformation and the quencher is separated from the fluorophore, and the fluorophore fluoresces. In the open conformation the initiator may be exposed such that it can interact with a sensor.

In a fifth aspect provided herein is a transmembrane nanosensor system in which the initiator interacts with a sensor to produce and optionally amplify a signal that can be used to identify exosomes with an open conformation of the DNA tweezers. The initiator may be paired with two hairpin nucleic acids to form a hybridization chain reaction and amplify the signal produced by the system.

### BRIEF DESCRIPTION OF THE DRAWINGS

FIG. 1A-F. A de novo designed Transmembrane Nano-Sensor (TraNS). (A) Four DNA strands self-assemble to form two reconfigurable arms TraNS device in its closed form. The two arms are connected at a hinge location that bears two hydrophobic cholesterol molecules (purple) that helps it insert spontaneously through lipid membrane. The external region (orange) of the device is 2× longer the internal region (blue). A molecular beacon hairpin (green) containing a fluorophore (orange sphere) and a quencher (black sphere) keeps the TraNS device in its closed form. (B) Upon reacting to a DNA target inside the membrane, the TraNS device reconfigures to its open form where the fluorophore goes far from the quencher and as a result the fluorescence increases, thereby transducing biochemical information across membrane. (C) Chemical structure of the cholesterol molecule, internally attached to the TraNS. (D) 10% Native PAGE gel analysis of the formation of unmodified TraNS. Lanes 1-4: 4 component strands, lane 5: 1T4Bp-key, lane 6-9: partially formed TraNS. Lane 6: 1+2, lane 7: 1+3, lane 8: 2+4, lane 9: 3+4, lane 10: TraNS-closed, lane 11: TraNS-open. The TraNS forms in quantitative yield and the open configuration being structurally less compact than the closed, runs slower in the gel. (E) Fluorescence spectra shows ˜11 fold increase in fluorescence upon structural reconfiguration of TraNS closed to TraNS-open. (F) Time dependent of fluorescence spectra shows kinetics of TraNS-closed to 1T4Bp-key target DNA (cyan) and RNA (blue). The binding of RNA is faster compared to DNA due to favorable thermodynamics of DNA-RNA bond formation compared to DNA-DNA.

FIG. 2A-F. Transmembrane signal transduction by membrane spanning TraNS without material transport (A) Experimental scheme to illustrate insertion and biosensing of TraNS device. When cholesterol modified TraNS inserts in small unilamellar vesicles (SUVs) filled with correct target, insertion of the TraNS device followed by binding to the target leads to structural reconfiguration of TraNS across the membrane—resulting in higher fluorescence (top, right). Absence of correct target inside SUVs does not cause structural reconfiguration, despite insertion of cholesterol modified structure (top, left). Non-cholesterol modified structures do not insert through the membrane and hence does no structural reconfiguration in presence of correct target (bottom, right) or random target (bottom, left) inside SUVs. (B) Histogram summarizing fluorescence signal obtained from TraNS+SUV samples as demonstrated in FIG. 2A. Data represent average fluorescence and standard deviation from three independent experiments. (C) Confocal images of giant-unilamellar vesicles (GUVs) filled with 1T4Bp-target+cholesterol modified TraNS show bright GUVs due to insertion of TraNS and detection of target (D) Confocal images of giant-unilamellar vesicles (GUVs) filled with 1T4Bp-target+unmodified TraNS show dark GUVs due to lack of insertion of TraNS. Scale bar—20 μm (E) Scheme illustrating the sulforhodamine-B (SRB) leakage assay to examine if transmembrane material transport. Formation of transmembrane pore (by alpha-hemolysin pore, for example) releases self-quenched SRB dye from SUVs, resulting in fluorescence enhancement. If, TraNS-mediated signal transduction happens in a material transport independent way—then no leakage of SRB happens, resulting in unenhanced signal. (F) Histogram representing average fluorescence and standard deviation from three independent experiments. 1% Triton X-100 detergent or alpha-hemolysin leads to leakage of SRB from SUVs—resulting in fluorescence increase compared to SUV only. Three versions of cholesterol modified TraNS does not show any fluorescence enhancement, demonstrating lack of material transport through TraNS.

FIG. 3A-F. TraNS inserts vertical to the membrane plane. (A) and (B) Scheme illustrating the assay used to elucidate the membrane interaction orientation of TraNS. If environment sensitive IANBD dye resides within the lipid membrane then its fluorescence is enhanced (A) compared to if it resides in water medium outside membrane (B). (C) Chemical structure of the IANBD molecule (green), internally labelled in designated locations of TraNS helices by reacting to phosphorothioate (PS) labelled (orange) DNA. (D) Locations of IANBD modification chosen to elucidate the orientation of the TraNS device with respect to the membrane. One location (L1) was chosen outside the membrane as a control and nine locations (L4, 9, 17, 34, 37, 42, 45, 50, 53) were chosen within the membrane at a span of 5.6 nm. From left to right SEQ ID Nos: 3, 35, and 8. (E) Direction of the nine intra-membrane locations chosen for IANBD modification. Considering 34.3°/bp rotation of B-DNA, the nine locations are distributed across the entire 360° around the two TraNS helices (red—right helix and blue—left helix). (F) Histogram representing the difference of IANBD fluorescence intensity of IANBD modified TraNS between buffer media and in SUVs—separately for 10 chosen locations. Large increase of fluorescence intensity upon membrane binding for the nine intramembrane locations compared to the external location I1 is consistent with our hypothesized perpendicular orientation of TraNS device upon insertion into membrane.

FIG. 4A-D. Cancer specific micro-RNA sensing by TraNS. (A) and (D). Fluorescence spectra of miR21-targetting TraNS-miR21 (A) and miR23b-targetting TraNS-miR23b (D) in closed (black), miR21 mediated open (blue—FIG. 4A) and miR23b mediated open (red—FIG. 4D) shows structural reconfiguration of TraNS-miR21 and TraNS-miR23b sensors by their respective targets. (B) Histogram of average fluorescence intensity maxima representing specificity of TraNS sensors toward other cancer micro-RNAs. TraNS-miR21 shows only signal enhancement for correct target (blue bar for miR21) but not for the incorrect targets. (C) Histogram summarizing fluorescence signal obtained from TraNS+target-filled SUV samples for TraNS-miR21. Only cholesterol modified TraNS shows fluorescence increase (blue bar for miR21) but not the unmodified negative control. Error bars derived from n=3 independent experiments.

FIG. 5. Schematic illustration of biomarker exosomal micro-RNA (ex-miR) sensing by cholesterol modified TraNS in intact exosomes. Cholesterol modified TraNS-miR21 (left) and TraNS-miR23b (middle) inserts through membrane of non-small cell lung cancer cell derived exosomes and reports the presence of their respective targets inside exosomes by transmembrane reconfiguration and consequent fluorescence enhancement. Lack of target miRs in healthy donor serum derived exosomes (right) does not lead to similar fluorescence enhancement.

FIG. 6A-C. Bench-top DNA origami nanoarray fabrication. (A) A comparison between the ease of counting individual fluorescent events when using randomized immobilization of origami (and other) single molecules versus their programmed placement at the diffraction-limit. (B) Schematic illustration of the DNA origami nanoarray patterning process which proceeds through 2D nanosphere close-packing, selective passivation, lift-off, and finally, Mg2+-mediated programmed origami placement. (C) Scanning electron micrographs of nanosphere close-packing (top view, and cross-section), and Atomic force micrographs of binding sites, and micro-scale origami placement analogous to schematic depiction (B) of process steps.

FIG. 7A-E. Enzyme-free, isothermal amplification paradigm for target amplification in a linear fashion (˜1 fluorophore every 2 minutes). (A) Schematic representation of the strand displacement reaction through cyclical displacement of the hairpins on detection of a target (initiator, I1) strand. (B) On-chip, in situ HCR on DON with each origami hosting a target-binding sequence. (C) As target (initiator, I1) concentration decreases, there is a reduction in total intensity in the analog format whereas digital detection is realized through the progressively lower counts of target binding events on the nanoarray. Scale bars=1 μm. Robustness validation with (D) 50% serum spiked initiator solutions, and (E) ˜200 random staple sequences. The limit of detection was ˜25 pM in each case. All error bars are SEM.

FIG. 8. Ex-miRNA detection at the single-exosome level. Anti-CD81 is patterned on each DNA origami. Exosomes from biofluids bind to the DON through interaction with anti-CD81 (dark blue) on the DNA origami and CD81 on the exosomal membrane (dark blue). Upon addition of TraNS, only target ex-miR containing exosomes light up (green spots).

### DETAILED DESCRIPTION

Described herein are methods and compositions for diagnosing or prognosing a disease using transmembrane nanosensors described herein. Also described are methods and composition for diagnosing or prognosing a disease using exosomes from a liquid biological sample from a subject.

One of the most important features of life is compartmentalization in which lipid bilayer membranes physically decouple chemical processes on either side of the bilayer¹. For these compartments to work in sync, it is essential to transfer chemical information across the bilayer. This is known as signal transduction² that allows cells to respond to their external environment and communicate with neighboring cells. The three main classes of protein machineries that are involved in the transmembrane signal transduction are—ligand gated ion channels³, receptor tyrosine kinases⁴ and most abundantly, G-protein coupled receptors (GPCRs)⁵. De novo engineering of the cellular signal transduction machinery with artificially designed molecular devices has enormous potential for applications in artificial tissue signaling, controlled drug delivery and biosensing. Various approaches have been taken to mimic ion channel proteins^{6,7} and receptor tyrosine kinases^{8,9}. In fact, the functional principle of ion channels has been utilized in commercial DNA sequencing¹⁰ and single molecule biosensing¹¹.

GPCRs are the most abundant signal transduction proteins that are targets of ˜50% of drugs currently on the market¹². Mimicking the transmembrane signal transduction mechanism of GPCR proteins has scope for development of artificial signal transduction among cell-like compartments¹³ for the creation of artificial tissues¹⁴. Furthermore, it has promise to enable unique opportunities such as sensing membrane enclosed biomarker without destroying the membrane. This can be greatly beneficial for simpler and cheaper ways of disease diagnostics. Despite enormous potential, it has been challenging to mimic the mechanism of GPCRs unlike other membrane proteins such as ion channels. Ligand binding to GPCRs on the extracellular part of the membrane leads to transmembrane conformational changes which relay the biochemical information to the cytosolic region¹⁵. This transmembrane structural reconfiguration along with proper orientation of the ligand binding and allosteric ends of the GPCRs on two sides of the membrane is crucial criteria for their function^{15,16}. Only two efforts have been reported till now for designing GPCR-mimetic synthetic molecules^{16,17}. In both cases, the GPCR-mimetic synthetic molecules only partially span the bilayer. Hence, signal transduction across the bilayer was unachievable by these molecules unlike GPCRs. Moreover, the small molecule ligands demonstrated there are highly specialized, proof-of-concept moieties that lack biological significance. A transmembrane sensor, long enough to probe both sides of the bilayer and capable of structural reconfiguration upon binding to a biologically relevant biomolecule will be—a) closer to the natural signal transduction proteins and b) will enable opportunities in biosensing of membrane enclosed biomarkers.

DNA nanotechnology¹⁸ offers great advantage for design of almost arbitrarily shaped nanostructures that can mimic biological systems with high structural precision and functional diversity^{19,20}. Membrane-binding DNA nanostructures that mimic transmembrane ion channels²¹⁻²⁶, membrane sculpting proteins²⁷, lipid scramblase²⁸ have been demonstrated. Moreover, various dynamically reconfigurable DNA nanostructures have been demonstrated previously^{20,29-33}. This makes DNA nanotechnology a suitable technique to design a dynamically reconfigurable transmembrane nanostructure that can mimic the functions of the GPCRs even closer to the natural system.

The ability to sense membrane enclosed nucleic acid biomarker without destroying the membranous vesicles enables unique opportunity for disease relevant biomarker sensing. Recently, cancer-associated micro-RNA biomarkers enclosed inside cell secreted lipid vesicles, namely exosomes^{34,35} have gained considerable attention for liquid biopsy-based diagnosis and prognosis of cancer^{36,37}. Current methods of exosomal micro-RNA (ex-miR) detection are difficult for point-of-care friendly clinical translation due to tedious, multistep and resource-heavy procedures involving ex-miR extraction, amplification and detection³⁸. A one step method that can detect ex-miRs without the need of extraction and amplification will simplify ex-miR based liquid-biopsy methods. Moreover, development of such method can enable unique opportunities such as exosome sorting³⁹ based on internal ex-miR content, studying inter and intra-exosome ex-miR heterogeneity⁴⁰. To this goal, we demonstrate the ability of our ex-miR targeting TraNS device to directly sense ex-miRs in intact exosomes without ex-miR extraction and amplification. By changing the sequence of the hairpin sensor region, we create a TraNS device that is designed for the detection of miR21, an ex-miR involved in various types of cancers⁴¹⁻⁴³. We show that our TraNS device can distinguish between healthy donor serum derived and cancer cell derived exosomes for presence of miR21.

Disclosed herein is a dynamically reconfigurable DNA nanostructure that spans the entire width of a membrane bilayer and transduces biochemical information across the membrane like GPCR proteins. Also disclosed herein is a demonstration of the applicability of this biomimetic approach in simplifying exosome based liquid biopsy diagnosis and prognosis.

### Definitions

The present invention is described herein using several definitions, as set forth below and throughout the application.

As used in this specification and the appended claims, the singular forms “a,” “an,” and “the” include plural references and, unless clearly indicated to the contrary, should be understood to mean “at least one.” Any reference to “or” herein is intended to encompass “and/or” unless otherwise stated.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term. In some aspects the term “about” means within 5% of a stated amount or concentration range or within 5% of a stated time frame.

It should also be understood that, unless clearly indicated to the contrary, in any methods claimed herein that include more than one step or act, the order of the steps or acts of the method is not necessarily limited to the order in which the steps or acts of the method are recited.

The term “liquid biological sample” as used herein will be understood to refer to a sample of biological fluid. Liquid biological samples include, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, vaginal secretions, nasal secretions, cerebrospinal fluid (CSF), urine, feces, sputum, saliva, nasal mucous, prostate fluid, lavage, semen, lymphatic fluid, bile, tears, sweat, breast milk, breast fluid, the like or combinations thereof. In some cases, the liquid biological sample is prepared by removal of cells from a blood sample. Liquid biopsy methods are described in the art. See, for example, WO2018093898, which is incorporated herein by reference in their entirety.

The liquid biological sample can be obtained from or provided by a subject by any appropriate means. As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “subject” is used herein interchangeably with “individual” or “patient.”

As used herein, the terms “synthetic” and “engineered” are used interchangeably and refer to the aspect of having been manipulated by the hand of man.

As used herein, the terms “disease”, “disease state”, and “disorder” will be understood to include, but not be limited to, any acute or chronic pathological condition which could benefit from diagnosis and/or treatment. Accordingly, this disclosure provides nucleic acid sensors and methods of using such nucleic acid sensors to detect a nucleic acid sequence associated with a disease (e.g., a cancer) in a liquid biological sample of a subject (e.g., human) for the purpose of diagnosing and/or treating the disease. In some embodiments, the cancer comprises non-small cell lung cancer.

### Polynucleotides

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of 5, 10, 15, 20, 30, 40, 50, or 100 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated. Purity may be denoted by a weight by weight measure and may be determined using a variety of analytical techniques such as but not limited to mass spectrometry, HPLC, etc.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

As used herein, the term “nucleotide” or “nucleotide moiety” refers to a sub-unit of a nucleic acid (whether DNA or RNA or analogue thereof) which includes a phosphate group, a sugar group and a heterocyclic base, as well as analogs of such sub-units. A “nucleotide monomer” refers to a molecule which is not incorporated in a larger oligo- or poly-nucleotide chain and which corresponds to a single nucleotide sub-unit. In some cases, other groups (e.g., protecting groups) can be attached to any component(s) of a nucleotide or nucleotide monomer.

A “nucleoside” or “nucleoside moiety” refers to a nucleic acid subunit including a sugar group and a heterocyclic base, as well as analogs of such sub-units. Other groups (e.g., protecting groups) can be attached to any component(s) of a nucleoside. A “nucleoside residue” refers to a molecule having a sugar group and a nitrogen containing base (as in a nucleoside) as a portion of a larger molecule, such as in a polynucleotide, oligonucleotide, or nucleoside phosphoramidite.

As used herein, the terms “nucleic acid polymer,” “nucleic acids,” of “polynucleotide” refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), as well as other hybridizing nucleic-acid-like molecules such as those with substituted backbones, e.g., peptide nucleic acids (PNAs) or other nucleic acids comprising modified bases and sugars. In some cases, the target nucleic acid is a double stranded DNA. In some cases, the target nucleic acid is cell-free DNA (cfDNA). However, the methods of the invention are not limited to double stranded DNA because other nucleic acid molecules, such as a single stranded DNA or RNA can be turned into double stranded DNA by one of skill in the arts using known methods. Suitable double stranded target DNA may be a genomic DNA or a cDNA.

Nucleic acids and/or other moieties of the invention may be isolated. As used herein, “isolated” means to separate from at least some of the components with which it is usually associated whether it is derived from a naturally occurring source or made synthetically, in whole or in part.

Nucleic acids and/or other moieties of the invention may include natural and/or non-natural bases.

As used herein, “modifying” (“modify”) one or more target nucleic acid sequences refers to changing all or a portion of a (one or more) target nucleic acid sequence and includes the cleavage, introduction (insertion), replacement, and/or deletion (removal) of all or a portion of a target nucleic acid sequence. All or a portion of a target nucleic acid sequence can be completely or partially modified using the methods provided herein. For example, modifying a target nucleic acid sequence includes replacing all or a portion of a target nucleic acid sequence with one or more nucleotides (e.g., an exogenous nucleic acid sequence) or removing or deleting all or a portion (e.g., one or more nucleotides) of a target nucleic acid sequence. Modifying the one or more target nucleic acid sequences also includes introducing or inserting one or more nucleotides (e.g., an exogenous sequence) into (within) one or more target nucleic acid sequences.

As used herein the term “lipid bilayer” or “phospholipid bilayer” refers to a thin polar membrane made of two layers of lipid molecules. These membranes are typically flat sheets that form a continuous barrier around cells, organelles, and other intra- and extracellular structures, such as exosomes. The cell membranes of almost all organisms and many viruses are made of a lipid bilayer, as are the nuclear membrane surrounding the cell nucleus, and membranes of membrane bound organelles in the cell. The lipid bilayer is the barrier that keeps ions, proteins, and other molecules where they are needed and prevents them from diffusing into areas where they should not be.

As used herein the term “exosome” is used interchangeably with “extracellular vesicle,” and refers to a cell-derived small (between about 30-100 nm in diameter) vesicle comprising a membrane that encloses an internal space, and which is generated from the cell by direct plasma membrane budding or by fusion of the late endosome with the plasma membrane. Exosomes have a characteristic lipid bilayer which has an average thickness of about 5 nm. Exosomes (extracellular vesicle), transport signaling proteins, nucleic acids, and lipids among cells. They are actively secreted by almost all types of cells, exist in body fluids, and circulate in the blood. Extracellular vesicles (EVs), exosomes, in particular, have gained attention for their potential as cancer biomarkers for liquid biopsies and readily available in all types of body fluids with high abundance (8-500 thousands/μL). Exosomes are stable membranous vesicles, released by the endocytotic pathway from a wide range of cells including cancer cells. Tumor exosomes are known to contain a plethora of information about tumor pathology and physiology and play a key role in cell-to-cell communication, by transferring bioactive molecules and oncogenic traits from cancer cells to normal cells through the delivery of oncogenic proteins, miRNAs, and mRNAs. Exosome cargo is cell-specific, suggesting that they can provide a unique signature of metastatic progression and the metabolic status of the tumor. Recent studies have uncovered evidence that miRNAs are transported in body fluids within exosomes, and once released into the extracellular space, they fuse with recipient cells and transfer their cargo making these miRNAs a non-invasive method to detect cancer progression and efficacy of therapy. Moreover, information about levels of specific ex-miRs have been reported to be indicative in predicting disease pathogenesis and hence valuable for prognosis. Hence, compositions and methods for rapidly and cost-effectively detecting and quantifying ex-miR from biofluids, such as the compositions and methods disclosed herein, present an attractive option for clinical translation of liquid biopsy-based cancer diagnosis and prognosis.

### Transmembrane Nanosensor (TraNS)

In some aspects, provided herein is a transmembrane nanosensor. In some embodiments, the transmembrane nanosensor includes a lipid conjugated DNA tweezer, a detectable marker or reporter molecule (e.g., a fluorophore, and a quencher suitably paired with the fluorophore). When the transmembrane nanosensor is in its un-bound state, the DNA tweezer is closed and the quencher and fluorophore are adjacent. When the target polynucleotide (i.e., the trigger strand) is bound to the hairpin loop, the DNA tweezer is in an open conformation, the quencher is separated from the fluorophore, and fluorescence from the fluorophore can be measured and quantified.

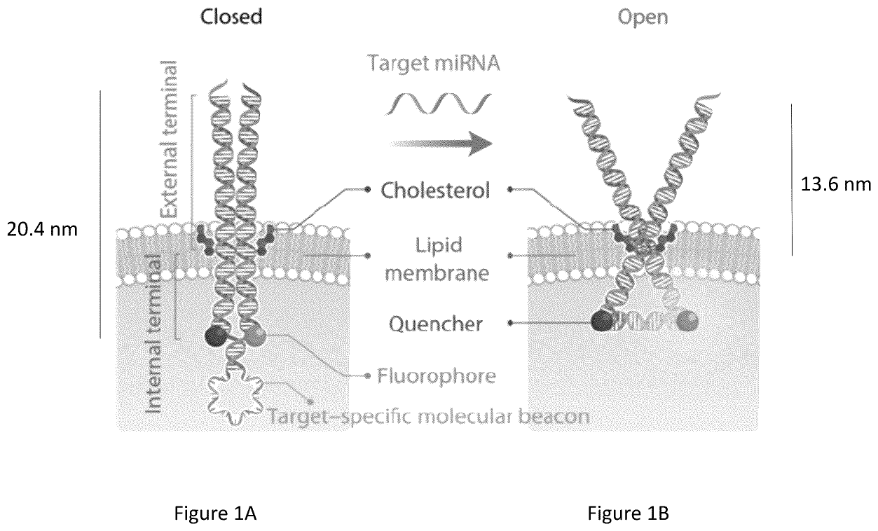

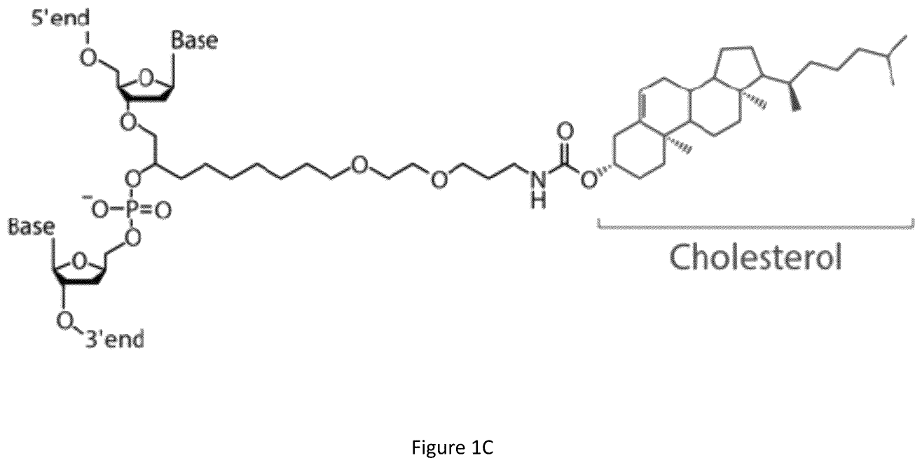

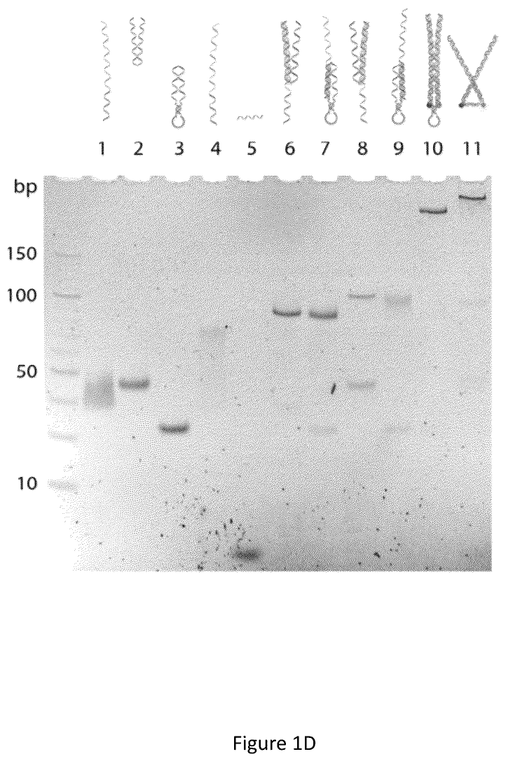

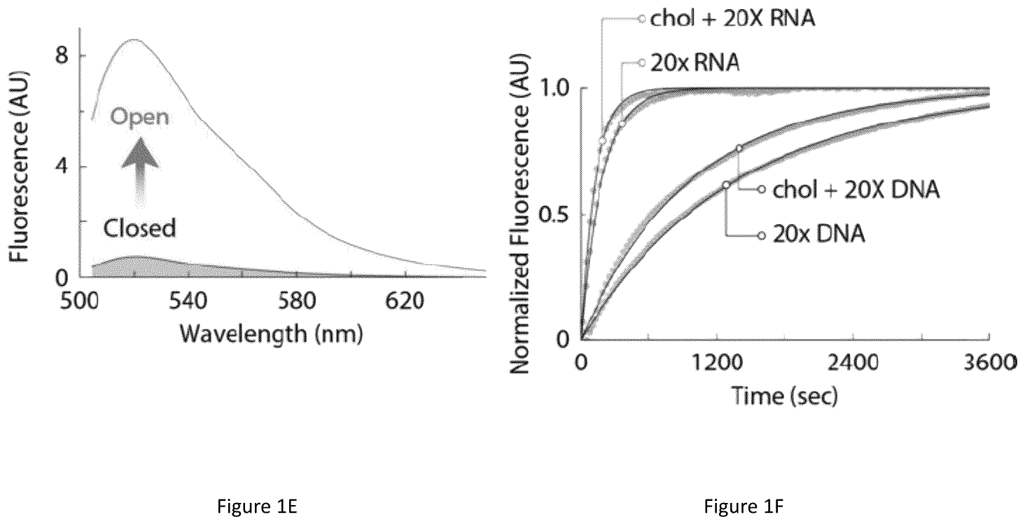

As used herein, “DNA tweezer” and “DNA nano-tweezer” are used interchangeably and refer to a nanoscale structure including a hairpin with a single-stranded loop and a first arm and a second arm linked by a crossover hinge wherein the distance between the tip of the first arm and the tip of the second arm is reversibly or irreversibly controlled by binding and release of a trigger strand to the single-stranded loop of the hairpin. In embodiments described herein, the trigger strand is external to the DNA nano-tweezer and is a target polynucleotide of interest. It will be readily understood by one of ordinary skill in the art that the flexibility and size of the DNA nano-tweezer may be manipulated by changing the size and sequences of DNA used in constructing the DNA nano-tweezer. In some embodiments, the first arm and second arm are double-crossover tile arms. In some embodiments, a more ridged multi-helix origami assembly may be utilized. One embodiment of a DNA nano-tweezer in both the closed and open conformation is depicted in FIGS. 1A and 1B. Conventional DNA nano-tweezer structures are known in the art. See for example Liu et al. (“A DNA tweezer-actuated enzyme nanoreactor,” Nature Communications, 2013, 4:2127); Zhou et al. (“Reversible regulation of protein binding affinity by a DNA machine,” J. Am. Chem. Soc., 2012, 134(3), 1416-1418); and U.S. application Ser. No. 16/653,235. Exemplary DNA tweezers of the present technology are shown in FIGS. 1A and 1B, in the closed and open configuration, respectively. Exemplary nucleic acid sequences of a DNA tweezer of the present technology are also provided below in Table 1.

In some embodiments, the DNA nano-tweezer disclosed herein comprises, in addition to the linked lipid molecules, one or more of: unique sequences or subsequences, unique overall length, one or more unique arm sequences, unique arm length(s), or a unique arm length ratio compared to prior art structures. With respect to arm length ratio, as exemplified in FIG. 1, the TraNS can be divided into two sections (the first arm set and the second arm set) as defined by the position of the hinge region and/or the lipid (e.g., cholesterol) molecules. By way of example, in some embodiments, the ratio of the arm length of the first arm set compared to the arm length of the second arm set is about 1:2. In some embodiments, the shorter arm set comprises the internal terminal (intermembrane portion) and the longer arm set comprises the external terminal (extramembrane portion), as shown in FIG. 1A.

In some embodiments, the hinge region comprises 1, 2, 3, or 4 lipid (e.g., cholesterol) molecules, and in some embodiments, the hinge region comprising the cholesterol molecules is embedded in an exosome lipid bilayer. While cholesterol is exemplified, other hydrophobic moieties would also facilitate the insertion into the lipid bilayer.

Table 1 below provides the SEQ ID NO., chemical modifications, and sequences of exemplary DNA oligonucleotides used to prepare various versions of the TraNS device. For IANBD modifications, the position of the modification is shown with an asterisk (*).

TABLE 1
Exemplary sequences for TraNS device

| SEQ ID NO: | Name | Sequence (5′ to 3′) |
|---|---|---|
| 1 | 1 | TTTTTGGCCCGCCGCAGTATACAACCTGACCAATAACAACGCGGAGAGCGGGTACAT |
| | | GCCTGGTCCACGTTTTT |
| 2 | 2 | CGTGGACCAGGCATGTACCCGCTCTCCGCGTTGTTATTGGTCACCGGTTGTGGAGCC |
| | | GCGGTTCGTACCTTCGGTGTAGCTCGGG |
| 3 | 4 | TTTTTCCCGAGCTACACCGAAGGTACGAACCGCGGCTCCACAACCGGACACAATCGA |
| | | TTTTTCGGTCGTTTTT |
| 4 | 3-key | TCGATTGTGTGGTTGTATACTGCGGCGGGCCTGCGG[T(BHQ-1)]AAGACCCACAA |
| | | [T(FAM)]CCGCTCGACCGAAAAA |
| 5 | 3-key-no dye | TCGATTGTGTGGTTGTATACTGCGGCGGGCCTGCGGTAAGACCCACAATCCGCTCGA |
| | | CCGAAAAA |
| 6 | 3-miR21 | TCGATTGTGTGGTTGTATACTGCGGCGGGCCTCAGAC[T(FAM)]CAACATCA |
| | | [T(BHQ-1)]GTCTGACGACCGAAAAA |
| 7 | 3-miR23b | TCGATTGTGTGGTTGTATACTGCGGCGGGCCTGG[T(BHQ-2)]AATCCCTGGCAAT |
| | | GTGATGAT[T(CAL Fluor Red 635)]ACCTCGACCGAAAAA |
| 8 | 1-Chol1 | TTTTTGGCCCGCCGCAGTATACAACCT[CholTEG]GACCAATAACAACGCGGAGAG |
| | | CGGGTACATGCCTGGTCCACGTTTTT |
| 9 | 1-Chol2 | TTTTTGGCCCGCCGCAGTATACAA[CholTEG]CCT[CholTEG]GACCAATAACAA |
| | | CGCGGAGAGCGGGTACATGCCTGGTCCACGTTTTT |
| 10 | 4-Chol1 | TTTTTCCCGAGCTACACCGAAGGTACGAACCGCGGCTCCACAACCG\|CholTEG]GA |
| | | CACAATCGATTTTTCGGTCGTTTTT |
| 11 | 4-Chol2 | TTTTTCCCGAGCTACACCGAAGGTACGAACCGCGGCTCCACAACCG[CholTEG]GA |
| | | C[CholTEG]ACAATCGATTTTTCGGTCGTTTTT |
| 12 | 1T4Bp-key | AGCGGATTGTGGGTCTTACCGCA |
| 13 | miR21-5p | TAGCTTATCAGACTGATGTTGA |
| 14 | miR23b-3p | ATCACATTGCCAGGGATTACC |
| 15 | 2-L1 | C*GTGGACCAGGCATGTACCCGCTCTCCGCGTTGTTATTGGTCACCGGTTGTGGAGC |
| | | CGCGGTTCGTACCTTCGGTGTAGCTCGGG |
| 16 | 2-L34 | CGTGGACCAGGCATGTACCCGCTCTCCGCGTTGT*TATTGGTCACCGGTTGTGGAGC |
| | | CGCGGTTCGTACCTTCGGTGTAGCTCGGG |
| 17 | 2-L53 | CGTGGACCAGGCATGTACCCGCTCTCCGCGTTGTTATTGGTCACCGGTTGTGG*AGC |
| | | CGCGGTTCGTACCTTCGGTGTAGCTCGGG |
| 18 | 2-L37 | CGTGGACCAGGCATGTACCCGCTCTCCGCGTTGTTAT*TGGTCACCGGTTGTGGAGC |
| | | CGCGGTTCGTACCTTCGGTGTAGCTCGGG |
| 19 | 2-L50 | CGTGGACCAGGCATGTACCCGCTCTCCGCGTTGTTATTGGTCACCGGTTG*TGGAGC |
| | | CGCGGTTCGTACCTTCGGTGTAGCTCGGG |
| 20 | 2-L42 | CGTGGACCAGGCATGTACCCGCTCTCCGCGTTGTTATTGGTC*ACCGGTTGTGGAGC |
| | | CGCGGTTCGTACCTTCGGTGTAGCTCGGG |
| 21 | 2-L45 | CGTGGACCAGGCATGTACCCGCTCTCCGCGTTGTTATTGGTCACC*GGTTGTGGAGC |
| | | CGCGGTTCGTACCTTCGGTGTAGCTCGGG |
| 22 | 3-L9 | TCGATTGTG*TGGTTGTATACTGCGGCGGGCCTGCGGTAAGACCCACAATCCGCTCG |
| | | ACCGAAAAA |
| 23 | 3-L4 | TCGA*TTGTGTGGTTGTATACTGCGGCGGGCCTGCGGTAAGACCCACAATCCGCTCG |
| | | ACCGAAAAA |
| 24 | 3-L17 | TCGATTGTGTGGTTGTA*TACTGCGGCGGGCCTGCGGTAAGACCCACAATCCGCTCG |
| | | ACCGAAAAA |
| 25 | aTraNS-2 | CTCTCCGCGTTGTTATTGGTCACCGGTTGTGGAGCCGCGGTTC |
| 26 | aTraNS-5 | GTACCTTCGGTGTAGCTCGGGTGCGGTTTTTTTTTTTTTCCGCTCGTGGACCAGGCA |
| | | TGTACCCG |

TABLE 2
Different exemplary versions of TraNS and the strands used to synthesize them.

| Name of the TraNS Structure | Strands used (see Table 1) |
|---|---|
| Unmodified TraNS-closed. Target - 1T4Bp-key | 1, 2, 3-key, 4 |
| Unmodified TraNS-closed. Target - miR21 | 1, 2, 3-miR21, 4 |
| Unmodified TraNS-closed. Target - miR23b | 1, 2, 3-miR23b, 4 |
| TraNS with 2 cholesterol modifications and donor | 1-Chol1, 2, 3-key, 4-Chol1 |
| and quencher in the sensor. Target - 1T4Bp key | |
| TraNS with 2 cholesterol modifications and donor | donor1-Chol1, 2, 3-miR21, 4-Chol1 |
| and quencher in the sensor. Target - miR21-5p | |
| TraNS with 2 cholesterol modifications and donor | 1-Chol1, 2, 3-miR23b, 4-Chol1 |
| and quencher in the sensor. Target - miR23b-3p | |
| TraNS with 4 cholesterol modifications and donor | 1-Chol2, 2, 3-key, 4-Chol2 |
| and quencher in the sensor. Target - 1T4Bp key | |
| TraNS with 4 cholesterol modifications and donor | 1-Chol2, 2, 3-miR21, 4-Chol2 |
| and quencher in the sensor. Target - miR41-5p | |
| TraNS with 4 cholesterol modifications and donor | 1-Chol2, 2, 3-miR23b, 4-Chol2 |
| and quencher in the sensor. Target - miR43b-3p | |
| TraNS with 4 cholesterol modifications and | 1-Chol2, 2-L1, 3-key-no dye, 4-Chol2 |
| IANBD at L1. Target - 1T4Bp key | |
| TraNS with 4 cholesterol modifications and | 1-Chol2, 2-L34, 3-key-no dye, 4-Chol2 |
| IANBD at L34. Target - 1T4Bp key | |
| TraNS with 4 cholesterol modifications and | 1-Chol2, 2-L53, 3-key-no dye, 4-Chol2 |
| IANBD at L53. Target - 1T4Bp key | |
| TraNS with 4 cholesterol modifications and | 1-Chol2, 2-L37, 3-key-no dye, 4-Chol2 |
| IANBD at L37. Target - 1T4Bp key | |
| TraNS with 4 cholesterol modifications and | 1-Chol2, 2-L50, 3-key-no dye, 4-Chol2 |
| IANBD at L50. Target - 1T4Bp key | |
| TraNS with 4 cholesterol modifications and | 1-Chol2, 2-L42, 3-key-no dye, 4-Chol2 |
| IANBD at L42. Target - 1T4Bp key | |
| TraNS with 4 cholesterol modifications and | 1-Chol2, 2-L45, 3-key-no dye, 4-Chol2 |
| IANBD at L45. Target - 1T4Bp key | |
| TraNS with 4 cholesterol modifications and | 1-Chol2, 2, 3-L9, 4-Chol2 |
| IANBD at L9. Target - 1T4Bp key | |
| TraNS with 4 cholesterol modifications and | 1-Chol2, 2, 3-L4, 4-Chol2 |
| IANBD at L4. Target - 1T4Bp key | |
| TraNS with 4 cholesterol modifications and | 1-Chol2, 2, 3-L17, 4-Chol2 |
| IANBD at L17. Target - 1T4Bp key | |
| Unmodified aTraNS-closed | 1, aTraNS-2, 3-key, 4, aTraNS-5 |
| aTraNS-closed with 4 cholesterols | 1-chol2, aTraNS-2, 3-key, 4-chol2, aTraNS-5 |

The TraNS of the present disclosure is configured to traverse a lipid bilayer (e.g., an exosome membrane), and to comprise an extramembrane portion and an intermembrane portion. To traverse an exosome bilayer, a structure of about 15-40 nm, about 20-30 nm, about 21, 22, 22.5, 23, 24, 25, 26, 27, 28, 29, 30, 31, 32, 33, 34, 35, 36, 37, 38, 39, or about 40 nm. In some embodiments, the structure is 20.4 nm in length (e.g., 6 turns), comprising extramembrane domains of about 15-20 nm, such as 13.6 nm (e.g., 4 turns) and an intramembrane portion of about 5-10 nm, such as about 6.8 nm.

The DNA tweezer of the transmembrane nano-sensor (TraNS) of the present disclosure comprises a hinge region. The hinge region, formed, in some embodiments, by a Holliday junction in the DNA tweezer, facilitates a conformational change in the TraNS upon binding of the target (‘trigger’). In some embodiments, the junction is not at the center of the tweezer, thereby creating asymmetric arms. In some embodiments, the shorter set of arms is internal (e.g., inside the exosome), while the longer set of arms is outside of the exosome.

In some embodiments, the DNA tweezer is linked to one or more lipid molecules, for example, a cholesterol molecule. In some embodiments, the DNA tweezer is linked to one, two, three, or four, or two to four hydrophobic cholesterol molecules. An exemplary, non-limiting cholesterol molecule is provided in FIG. 1C. In some embodiments, the one or more lipid molecules are linked to the DNA tweezer at or near the hinge region. In some embodiments, the lipid molecule is positioned such that the extramembrane portion of the TraNS is about 2× or greater as long as the intramembrane portion (e.g., as shown in FIGS. 1A and 1B). While cholesterol is exemplified, other hydrophobic molecules, such as tocopherol, porphyrin, alkyl groups, lipid molecules would facilitate the insertion of sensors into the lipid bilayers. In some embodiments, the cholesterol molecules (or other hydrophobic molecules) are covalently linked into the strands constituting the TraNS device.

Linking chemistries are well known in the art, and modified nucleic acids (e.g., comprising linked cholesterol molecules or linked labels) are commercially available. Some considerations for the linking chemistry related to cholesterols include the use of a TEG spacer to a) properly present the cholesterol molecule away from the helices, and b) to keep the Chol-TEG phosphoramidites soluble in the aqueous during synthesis. While choosing the position of the fluorophore in the sensor region it is preferable to put them next to a non-guanine base to avoid proximal G-base quenching.

In some embodiments, the TraNS as disclosed herein comprises one or more detectable markers/reporters. In some embodiments, the marker is detected when the DNA tweezer is in a closed conformation. In some embodiments, the marker is detected when the DNA tweezer is in an open conformation. In some embodiments, the detectable marker comprises a quencher fluorophore pair, many of which are known and described in the art. In some embodiments, the quencher-fluorophore pair are suitable for fluorescence resonance energy transfer (FRET). Such markers are well known in the art. Exemplary, non-limiting markers include (1) FAM & BHQ-1 (2) Alexa488 & BHQ-1 (3) Alexa555 and BHQ-2 (4) Alexa532 and BHQ-1 (5) Quasar 570 and BHQ-2 (6) Cal Fluor Red 635 and BHQ 2. In some embodiments, the markers include FAM & BHQ-1; Quasar 570 & BHQ-2; and Cal Fluor Red 635 & BHQ 2. In some embodiments, the markers are positioned on the intramembrane portion or the extramembrane portion of the TraNS, for example, at the ends, or near the ends of the DNA tweezer arms (see e.g., FIGS. 1A and 1B).

The DNA tweezer of the TraNS comprises a hairpin loop. The hairpin loop is single stranded and comprises a region of complementarity to a target (trigger) sequence. Accordingly, each TraNS comprises a hairpin loop that is specific for a particular target. In other words, the sequence and structure of TraNS molecule may be identical, except for the hairpin loop. A hairpin loop is typically 15-40 nucleotides in length and exhibits a hybridization region of at least about 80-90%, 80-92%, 80-95%, 80-98% or 80-100% complementary to the target sequence. In some embodiments, there is 100% complementary between the target DNA or RNA with the complementary region of the hairpin loop. Exemplary hairpin loop complementary region sequences and their complementary target sequences include those shown in the table below. In addition to the complementary regions, the hairpin loop comprises about 3, 4, 5, 6,7, 8, 9,or about 10 extra nucleotides, on each side of the complementary region, to form a stem region to facilitate closing of the hairpin. In some embodiments, the hairpin comprises about 3-4 nt in the stem region to facilitate the closing of the hairpin

TABLE 3
Exemplary target-hairpin pairs

| Target sequence | Hairpin sequence |
|---|---|
| SEQ ID NO: 27: | SEQ ID NO: 31: |
| AGCGGATTGTGGGTCTTACCGCA | TGCGGTAAGACCCACAATCCGCT |
| SEQ ID NO: 28: | SEQ ID NO: 32: |
| TAGCTTATCAGACTGATGTTGA | TCAGACTCAACATCATGTCTG |
| SEQ ID NO: 29: | SEQ ID NO: 33: |
| ATCACATTGCCAGGGATTACC | TGGTAATCCCTGGCAATGTGATG |
| | ATT |
| SEQ ID NO: 30: | SEQ ID NO: 34: |
| ATCACATTGCCAGGGATTACC | TATTCCCCTAGAATCGAATCTGT |
| | GGGAATT |

As used herein, “closed conformation” refers to the conformation of the DNA nano-tweezer wherein the hairpin loop is free and unbound by a trigger strand. In the closed conformation, the distance between the tip of the first arm and the tip of the second arm is about 4 nm (e.g., 3, 4, 5, or 6 nm). In some embodiments, the distance between the tip of the first arm and the tip of the second arm in the closed conformation is between about 3 nm and about 18 nm, between 3 nm and 16 nm, between 4 nm and 14 nm, or between 4 nm and about 10 nm. In some embodiments, the distance between the tip of the first arm and the tip of the second arm is less than 18 nm, less than 17 nm, less than 16 nm, less than 15 nm, less than 14 nm, less than 13 nm, less than 12 nm, less than 11 nm, less than 10 nm, less than 9 nm, less than 8 nm, less than 7 nm, less than 6 nm, less than 5 nm, less than 4 nm, less than 2 nm, or less than 1 nm. The length of the arm and the position of the FRET pairs along the arm tune the distance between FRET donor and acceptor molecules As is known in the art, the Foster radius is on the order of 4 nm, ideal separation for high FRET is 2 nm, and in most cases, low FRET (not quenching) is achieved at 6 nm separation. Accordingly, and by way of example only, if a FRET pair is employed in the DNA tweezers of the present technology, in some embodiments, the closed conformation results in the FRET pair being separated by about 6 nm or less.

As used herein, “open conformation” refers to the conformation of the DNA nano-tweezer wherein the trigger strand is bound (e.g., base paired) to the hairpin loop. In the open conformation, the distance between the tip of the first arm and the tip of the second arm is about 16 nm (e.g., 12 nm, 13 nm, 14 nm, 15 nm, 16 nm, 17 nm, 18 nm, 19 nm, or 20 nm). In some embodiments, the distance between the tip of the first arm and the tip of the second arm in the open conformation is between about 12 nm and about 20 nm, between about 13 nm and about 19 nm, between about 14 nm and about 18 nm, or between about 15 nm and about 17 nm. In some embodiments, the distance between the tip of the first arm and the tip of the second arm in the open conformation is at least 12 nm, at least 13 nm, at least 14 nm, at least 15 nm, at least 16 nm, at least 17 nm, at least 18 nm, at least 17 nm, at least 20 nm, at least 30 nm, or at least 40 mn. Accordingly, and by way of example only, if a FRET pair is employed in the DNA tweezers of the present technology, in some embodiments, the open conformation results in the FRET pair being separated by 6 nm or greater.

In various embodiments of the DNA nano-tweezers described herein, binding of the trigger (target) to the hairpin loop results in an increase in the distance between the tip of the first arm and the tip of the second arm and increases the distance between the fluorophore and the quencher. The increase in distance between the fluorophore and the quencher may be an increase of about 4 nm, 6 nm, 8 nm, 10 nm, 11 nm, 12 nm, 13 nm, 14 nm, 16 nm, or more.

As used herein, “trigger strand” may be used interchangeably with the term “target” and refers to a nucleic acid oligonucleotide that is complementary to and binds to the hairpin loop of the DNA nano-tweezer to initiate a conformation change in the DNA nano-tweezer from the closed conformation to the open conformation. In the embodiments described herein, the trigger strand is a target polynucleotide of interest. In some embodiments, the trigger strand may be between about 14 bases and about 40 bases (e.g., 15 to 35 bases, 18 to 30 bases, 20 bases to 28 bases) in length. In some embodiments, the trigger strand is about 21 bases in length (e.g., 15 bases, 16 bases, 17 bases, 18 bases, 19 bases, 20 bases, 21 bases, 22 bases, 23 bases, 24 bases, or 25 bases). As used herein “trigger strand” is used interchangeably with “target” nucleic acid. The trigger strand may be DNA or RNA, and in some embodiments, is identified within an exosome.

### Methods

In some aspects, described herein is a method of diagnosing or providing a prognosis for a disease. In some embodiments, the method includes obtaining a liquid biological sample from a subject, extracting exosomes from the liquid biological sample, contacting the exosomes with a transmembrane nanosensor described herein, measuring and quantifying fluorescence from the transmembrane nanosensor, and diagnosing the subject with the disease or providing a prognosis. In some embodiments, the disease is a neurodegenerative disease (e.g., multiple sclerosis, Alzheimer's disease, etc.), diabetes, cardiovascular disease, and cancer. In some embodiments, the disease is cancer.

The terms “detect” or “detection” as used herein indicate the determination of the existence, presence or fact of a target or signal in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through visually detectable signals: fluorescence, spectra, or images from a target of interest or a probe attached to the target.

For the purposes of this disclosure, the term “target” refers to a nucleic acid molecule or polynucleotide that is the intended nucleic acid molecule to be detected by a method disclosed herein or to be bound by a transmembrane nanosensor as described herein (“trigger”). For example, the target may be a cancer specific exosomal micro RNA (miRNA) if the method is employed to detect the presence of cancer in a patient sample. In some embodiments, the target is an exosomal miRNA. In some embodiments, the target is a DNA. In some embodiments, the target is an RNA. The term “non-target” refers to a nucleic acid molecule or polynucleotide that is not intended to be detected by a method disclosed herein or to be bound by a transmembrane nanosensor as described herein.

In some embodiments, exosomes from a subject sample, e.g., a liquid biological sample such as a blood sample may be tested for the presence and/or quantity of a target molecule. In some embodiments, detection of the target is indicative of a disease or condition, such as cancer. By way of example but not by way of limitation, detection of miR-21-5p or miR-23b-3p in exosomes from a subject indicates the presence of cancer cells, such as non-small cell lung cancer. While two examples are provided herein, there are many published examples related to other exosome bound targets indicative of disease. For example, ExosomeDX has a diagnostics kit that targets exosomal RNA for prostate cancers. This diagnostics kit targets the gene signature of exosomal RNA of ERG, PCA3, SPDEF. For exemplary prostate cancer targets, see Table 2 of Zhan et al., Cancer Manag Res. 2018; 10:4029-4038, incorporated herein by reference in its entirety.

In some embodiments, the presence, absence, or level of single target is evaluated; in some embodiments, the presence, absence, or level of multiple targets are evaluated. For example, a first target may be detected by a first TraNS, comprising a hairpin loop specific to the first target and a probe or report specific to the first target. A second target is detected using a second TraNS wherein the second TraNS comprises a second hairpin loop that is specific for the second target, and a second probe or reporter. In some embodiments, the first and second probe/reporter are the same. In some embodiments, the first and second probe/reporter are different.

In some aspects, the exosomes may be contacted with the transmembrane sensor either in “free” form, e.g., in a test well or test tube, or in “bound” form, e.g., bound to a solid surface to form an exosome nanoarray. Immobilized nanoarrays are known and described in the art. See, for example, WO2019108954, which describes methods of making such nanoarrays and which is incorporated herein by reference as if set forth in its entirety.

### Nanoarrays

Despite their promise as biomarkers for detecting cancer progression and assessing efficacy of therapy, developing a clinical test for exosomal microRNA's (ex-miRs) based liquid biopsy methods has been challenging. Being encapsulated inside exosomes, the ex-miRs first need to be extracted from exosomes. This process makes it necessary to first isolate the exosomes from body fluids using time-consuming, tedious, low-yield methods such as ultracentrifugation, expensive exosome extraction kits etc. As a result, current technologies for ex-miR detection, such as qRT-PCR, or microarray screening test are expensive, slow, tedious, and require highly specialized skills and resources. Moreover, due to the need to lyse exosomes for ex-miR extraction, no current technology can quantify the relative abundance of ex-miR containing exosome subpopulation. Hence, development of a rapid, in situ, ultrasensitive detection and quantification of ex-miR in intact exosomes would facilitate clinical translation of ex-miR based liquid biopsy. This would require (i) a transmembrane sensor that can detect ex-miRs in situ without lysing exosomes and (ii) a platform that enables rapid, cheap, ultrasensitive quantification of the ex-miRs.

Disclosed herein are methods, compositions, systems, and platforms for ex-miR detection and quantification utilizing, in one embodiment, nanoarrays of antibodies and transmembrane sensors. DNA nanotechnology utilizes DNA as a building material to build precisely defined structures at nanometer scale resolution. Various sensors and amplification devices have been designed using the structural and functional properties of DNA. Molecular beacons (MBs) are a class of DNA based sensors that signals binding of target DNA or RNA via fluorescence enhancement. MBs have previously been used for miRNA sensing both in vitro and in vivo. However, the main challenge in detecting ex-miRs from intact exosomes is to deliver the MB through the exosome membrane. Membrane protein mimetic DNA nanostructures have been demonstrated previously that span lipid bilayer without disrupting it. Here, we incorporate an ex-miR-specific MB sensor to a membrane spanning DNA nanostructure to design a Transmembrane Nano-Sensor (TraNS) device that can sense ex-miR without lysing the exosomes. This significantly simplifies the ex-miR detection by circumventing the need of exosome isolation, exosome lysis, and RNA extraction. Nanoarray technology enables rapid, high-throughput and ultra-sensitive quantification by digital counting of signals compared to traditional analog measurements. Using our low-cost DNA Origami Nanoarray (DON) platform, we propose direct capture of exosomes on a highly patterned grid from biofluids and subsequent TraNS device based-sensing of the ex-miR content of these exosomes in high-throughput, ultra-sensitive and digitally quantitative way for the first time.

Disclosed herein is simple, rapid, sensitive and digitally quantitative method for ex-miR detection and quantification. 1) A transmembrane sensor that can detect ex-miR without disrupting the exosome membrane. 2) By integrating the transmembrane sensor on a patterned nanoarray provides an ex-miR based diagnostic method and platform that is rapid, cheap, ultrasensitive and digitally quantifiable.

Accordingly, in some embodiments, the exosomes are bound to a solid surface by an exosome specific antibody. In some embodiments, the exosome specific antibody is anti-CD8. While anti-CD8 is exemplified other non-limiting targets would include CD-3, CD-9, CD-63, EphA2. In general, the capture approaches could include other binders, such as peptides, nanobodies, aptamers, and somamers.

The solid surface may be any solid surface known in the art suitable for immobilization of an antibody or other macromolecule. Suitable solid surfaces include, but are not limited to glass, plastic, paper, silanol-enriched glass, a bead, a plate, a dish, or a microfluidic device. In some embodiments, the surface is coated with an antifouling film, such as PEG. For embodiments comprising optical readouts, transparent surfaces with the thickness that is less than the working distance of optical objectives are employed.

### Illustrative Embodiments

1. A transmembrane nanosensor device comprising a lipid conjugated DNA tweezer comprising a hairpin loop complementary to a target polynucleotide trigger strand; a fluorophore; and a quencher paired to the fluorophore, wherein when the hairpin loop is bound by the target polynucleotide trigger strand, the DNA tweezer transitions from a closed conformation to an open conformation, the quencher is separated from the fluorophore, and the fluorophore fluoresces.

2. The transmembrane nanosensor device of embodiment 1, wherein the lipid conjugated DNA tweezer is integrated into a lipid bilayer.

3. The transmembrane nanosensor of any of embodiments 1-2, wherein the target polynucleotide trigger strand is an RNA or a DNA polynucleotide.

4. The transmembrane nanosensor of any of embodiments 1-3, wherein the lipid is a cholesterol molecule.

5. The transmembrane nanosensor of any one of embodiments 1-4, wherein the open conformation of the DNA tweezer exposes an initiator sequence.

6. A transmembrane nanosensor system, comprising the transmembrane nanosensor of embodiment 5 and a first hairpin nucleic acid, a second hairpin nucleic acids, wherein the first and second hairpin nucleic acids are labeled with a fluorescent marker and a quencher such that in the hairpin configuration the fluorphore is paired with the quencher, wherein the initiator sequence is complementary to the 5′ end and 5′ stem of the first hairpin and the same sequence as the loop and the 3′ stem of the second hairpin, and wherein the first hairpin loop and 3′ end of the stem are complementary to the 5′end and the 5′ stem of the second hairpin.

7. The transmembrane sensor system of embodiment 6, wherein when the DNA tweezer transitions from a closed conformation to an open conformation the initiator is exposed and binds to the first hairpin and via strand displacement of the stem by binding to the initiator the fluorphore is separated from the quencher and the first hairpin binds to the second hairpin via strand displacement and the fluorphore of the second hairpin is separated from the second quencher.

8. A method of labeling an exosome comprising contacting the exosome with the transmembrane nanosensor of any of embodiments 1-7; and measuring and quantifying fluorescence of the transmembrane nanosensor, wherein fluorescence of the nanosensor indicates the presence of the target polynucleotide trigger in the exosome.

### EXAMPLES

The following examples are illustrative and should not be interpreted to limit the scope of the claimed subject matter.

### Example 1

### Biomimetic Transmembrane Signal Transduction and Detection of Cancerous Biomarkers from Liquid Biopsy Exosomes

### Abstract

Trans-membrane signal transduction by highly-evolved membrane proteins such as G-protein coupled receptors (GPCR's) play crucial role in regulating pivotal cellular decisions, such as proliferation, differentiation, metabolism and apoptosis. De novo engineering of cellular signal transduction machinery with artificially designed molecular devices has enormous potential in synthetic biology for application in artificial tissue signaling, biosensing, and controlled drug delivery. Here, we demonstrate Trans-membrane signal transduction by a completely synthetic self-assembled DNA nanostructure that inserts through lipid membrane and dynamically reconfigures upon sensing a membrane-enclosed DNA or RNA target, thereby transducing biomolecular information across lipid membrane. Our Transmembrane Nano Sensor (TraNS) device can detect nucleic acid biomarkers inside lipid vesicles without the need of destroying the membrane to extract the encapsulated biomarker. A key challenge in the field of exosomal micro-RNA (ex-miR) based liquid biopsy is the extraction and amplification of the ex-miR. We demonstrate that our biomimetic TraNS device can sense cancer associated ex-miR biomarkers inside exosomes without the need of RNA extraction and amplification.

### Design and Formation of Reconfigurable Nanosensor

We designed a Transmembrane Nano-Sensor (TraNS) able to insert itself through the lipid bilayer and dynamically reconfigure upon sensing a target molecule on one side of the membrane (FIGS. 1a & b), enabling transduction of biochemical information across bilayer as fluorescence signal. We rationalized that unlike previous ion channel mimetic DNA nanostructures^{21,23-27}, a transmembrane signal transducing nanostructure that mimics GPCR proteins should be free of leakage across membrane as GPCRs transduce signal by structural reconfiguration and not by material transport. Therefore, to keep TraNS device small and simple enough for transmembrane structural reconfiguration without too much membrane perturbation, we design it as a simple tile structure instead of scaffolded origami. Four individual DNA strands self-assemble to form two “arms” of the TraNS device, joined by a Holliday junction^{44,45} acting as a hinge. A single stranded DNA hairpin (FIGS. 1a & b, green region), containing an optimized stem-loop sequence^{31,46} bridges the two arms at the shorter end. In the absence of the target this exists as a hairpin, keeping the TraNS device closed. Upon binding to a target DNA or RNA, the hairpin extends to form a double strand. As a result, the TraNS device reconfigures around the hinge from a closed “H-like” shape (FIG. 1a) to an open “X-like” shape (FIG. 1b). A fluorophore-quencher pair (black and orange spheres in FIGS. 1a & b) is placed in the hairpin sensor in a molecular beacon fashion⁴⁷. Target binding spatially separates the fluorophore from the quencher, resulting in an increase of fluorescence intensity. Inspired from previous membrane-interacting DNA nanostructures⁴⁸, we decorated the TraNS device with two hydrophobic cholesterol molecules (FIG. 1c) at the hinge to facilitate its insertion into the lipid membrane. The cholesterol molecules are placed such that the extramembrane part is ≥2× as large compared to the intramembrane part as shown in (FIG. 1a-b) for preferential orientation of the TraNS sensors in the membrane.

Native poly-acrylamide gel electrophoresis (PAGE) shows successful formation of the TraNS device in stoichiometric yield and desired structural reconfiguration (FIG. 1d). The closed conformation being slightly more compact, runs faster in the gel compared to the open conformation³¹ (lanes 10 and 11). Steady state fluorescence spectra show ˜11-fold increase in fluorescence intensity upon interaction of unmodified TraNS device with target (FIG. 1e) due to spatial separation of quencher from the fluorophore upon device opening, confirming the desired structural reconfiguration. Time trace of fluorescence emission shows that the structural reconfiguration happens with DNA (FIG. 1f—cyan curve) as well as RNA (FIG. 1f—blue curve) target. Experiments with RNA molecules consistently give faster switching kinetics than identical assays with target DNA analogs.

### TraNS Insertion and Transmembrane Signal Transduction Across Intact Membrane

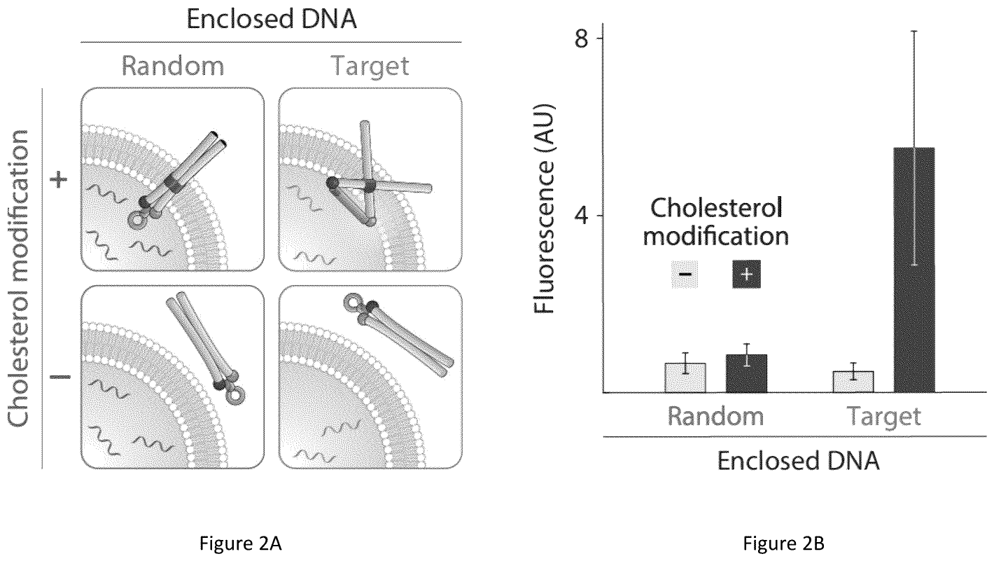

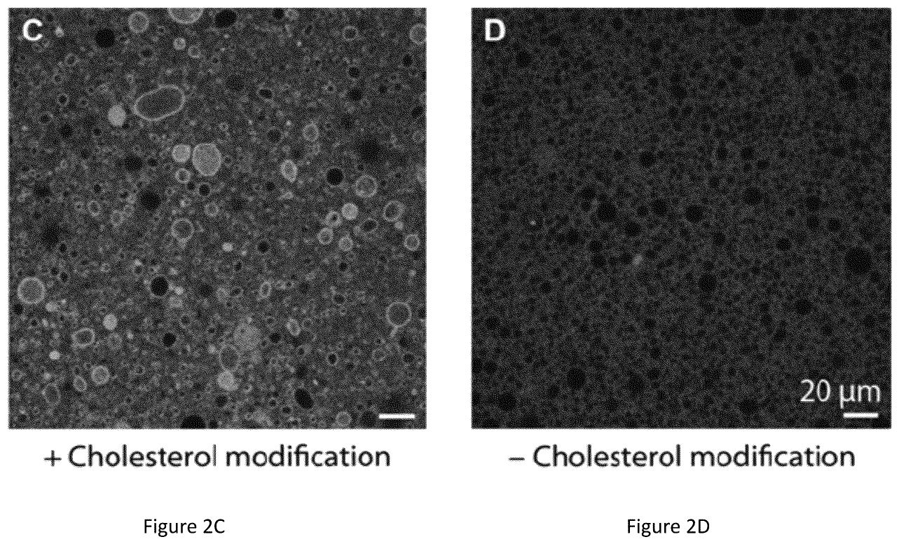

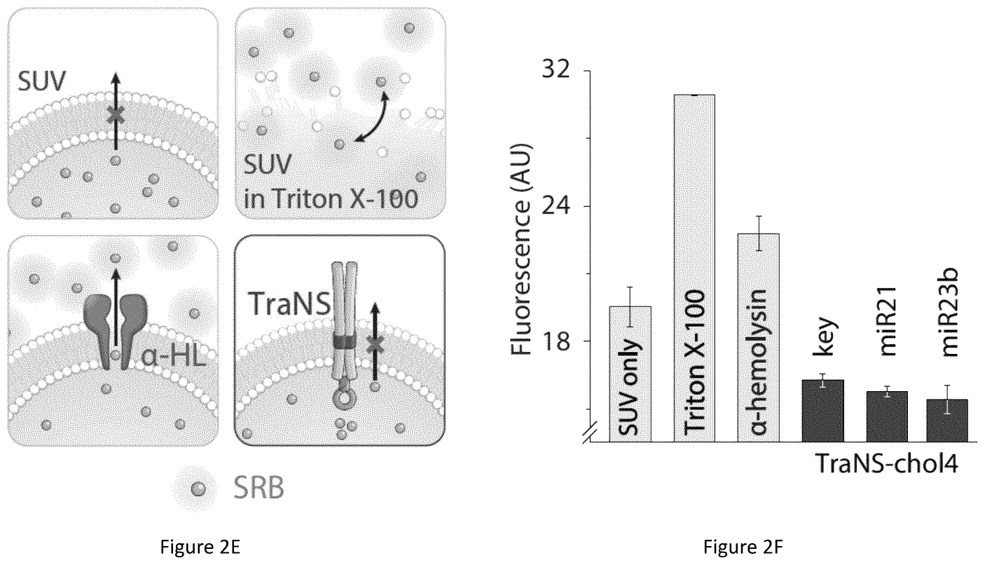

To test the insertion of cholesterol modified TraNS device in lipid membrane and transmembrane signaling across the membrane, we utilized its target mediated fluorescence enhancement property. We encapsulated target DNA in small unilamellar vesicles (SUVs) and added cholesterol modified TraNS to this. Fluorescence spectra show that the cholesterol-modified TraNS device selectively inserts through the membrane. (FIG. 2A). Upon binding to the membrane-enclosed target DNA, there is an increase in fluorescence (FIG. 2B) and bright GUVs (FIG. 2C) due to the membrane insertion and opening of TraNS by target DNA molecules. Negative controls with TraNS without cholesterol and SUVs with random DNA sequences lead to <0.1× fluorescence intensity (FIG. 2B) and dark GUVs (FIG. 2D), showing that cholesterol modified TraNS device inserts through the membrane and specifically signals the presence of membrane-enclosed target by transmembrane structural reconfiguration.

To verify whether TraNS-mediated signal transduction happens in a material transport independent manner similar to GPCRs and unlike ion channels, we tested whether insertion of cholesterol modified TraNS leads to outflux of small molecule fluorophore sulfo-rhodamine B (SRB). In the SRB release assay, commonly used to test pore formation ability of materials^{21,49-52} in lipid membrane, the SRB fluorophores are self-quenched at high concentrations (50 mM) inside the vesicles, but increase in emission upon release into the solvent (FIG. 2E). Insertion of cholesterol modified TraNS sensors, specific to 1T4Bp-key, micro RNA 21 (miR21) and micro RNA 23b (miR23b) into SRB-filled vesicle does not lead to SRB release (FIGS. 2E and 2F) unlike pore forming materials such as alpha-hemolysin or detergent (FIGS. 2E and 2F). Interestingly, we notice slight reduction of fluorescence in case of the TraNS devices. Insertion of TraNS into SUVs increases the local concentration of SRB by occupying some volume. As a result, enhanced self-quenching of SRB in the tightly sealed vesicles leads to reduction of fluorescence compared to blank control in FIG. 2F.

### Orientation of TraNS in Membrane

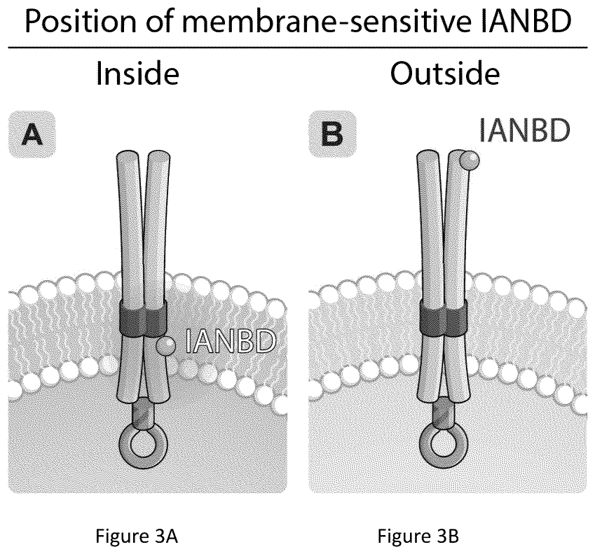

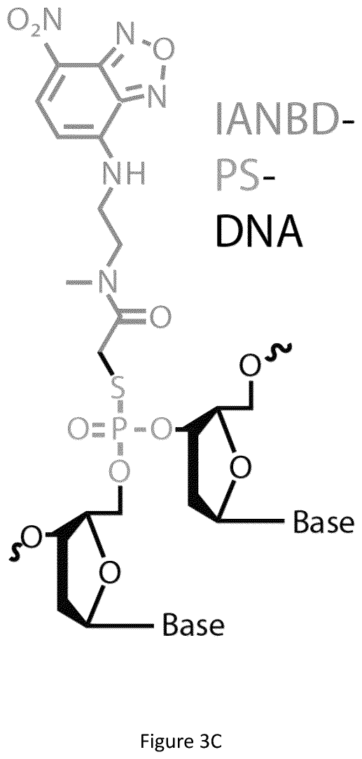

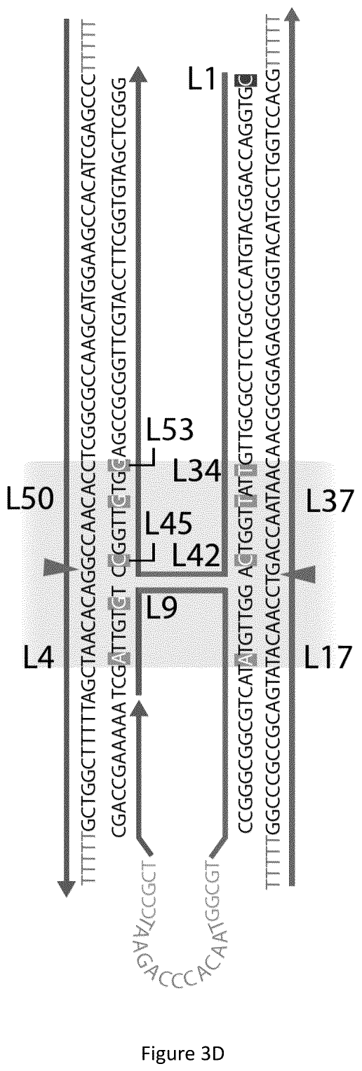

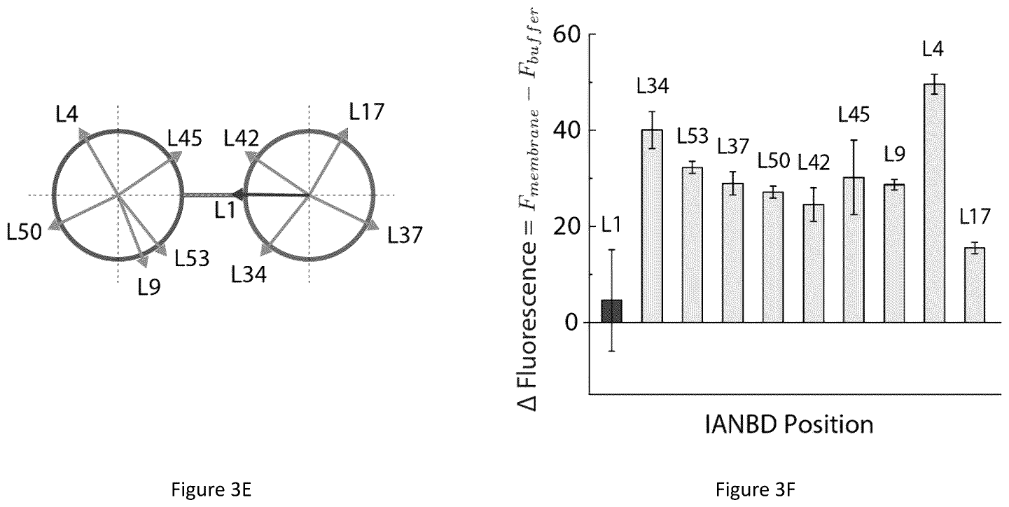

After validating insertion of TraNS device into the membrane, we investigated whether the device inserts normal to the membrane rather than lying flat on it. We hypothesized that—if the entire 360° of the membrane-associated regions of the TraNS remains inside the membrane, then it will prove that the TraNS inserts normal to the membrane. For this—we adopted an assay, extensively used to determine the membrane-spanning domains of transmembrane proteins and peptides^{53,54}. In this assay—a dye N,N′-dimethyl-N-(iodoacetyl)-N′-(7-nitrobenz-2-oxa-1,3-diazol-4-yl)pethylenediamine(IANBD) is used whose fluorescence intensity is strongly quenched in the presence of water and significantly enhances when it is moved to a non-aqueous medium. We rationalized that—positioning IANBD in a membrane-embedded location of TraNS would result in its fluorescence enhancement in lipid vesicles (FIG. 3A) compared to if it resides outside the membrane (FIG. 3B).

We conjugated⁵⁵ IANBD molecules (FIG. 3C) to the component DNA strands bearing phosphorothioate modification in designated locations. Assuming the position of cholesterol bearing nucleotides in the middle of the bilayer leaflet, we chose total 10 positions across the TraNS device—1 outside (L1) the membrane and 9 inside (L4, L9, L17, L34, L37, L42, L45, L50 and L53) the membrane (FIG. 3D), encompassing the entire 360° of the TraNS helices (FIG. 3E). For every location of IANBD modification, the IANBD characteristic fluorescence intensity of cholesterol and IANBD modified TraNS device is measured in buffer only and upon addition of SUVs. We noticed that the position outside the membrane (I1) showed very slight fluorescence enhancement in SUVs (FIG. 3F) as expected. However, 9 membrane-embedded positions (L4-L53) showed 6 to 12-fold more fluorescence enhancement in SUVs compared to that of I1 (FIG. 3F). This demonstrates that all the 9 positions, spanning almost the entire 360 degree are embedded in the membrane. Hence, we conclude that the TraNS device is inserted normal to the bilayer rather than lying flat on the bilayer surface. Although, previous membrane interacting DNA nanostructures were hypothesized to insert perpendicular to the membrane place but, this is the first time where such insertion orientation has been verified empirically.

### Cancer Specific Micro-RNA Sensing by TraNS

Micro-RNAs (miRs) are 19-26 nt long non-coding RNA molecules that have gained considerable interest due to their role in various cellular transformation and disease relevance. Especially, miRs inside cell secret lipid vesicles namely exosomes^{34,35} have gained considerable attention for liquid biopsy-based diagnosis and prognosis of cancer^{36,37}. We realized that exosomal miR specific TraNS will enable exosome enclosed miR sensing without the need of destroying the membranes. As a proof of concept, we chose two miRs—miR-21-5p and miR-23b-3p that have been found to be diagnostic and prognostic marker in non-small cell lung cancer (NSCLC)^{42,43}.

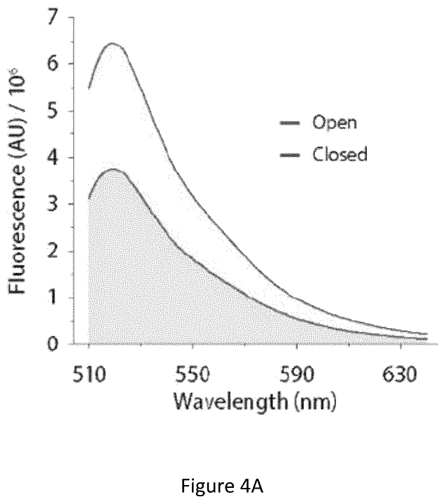

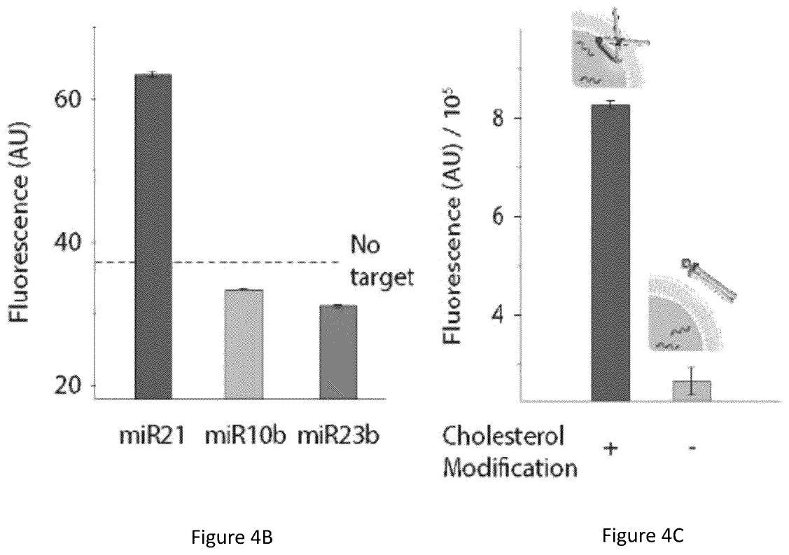

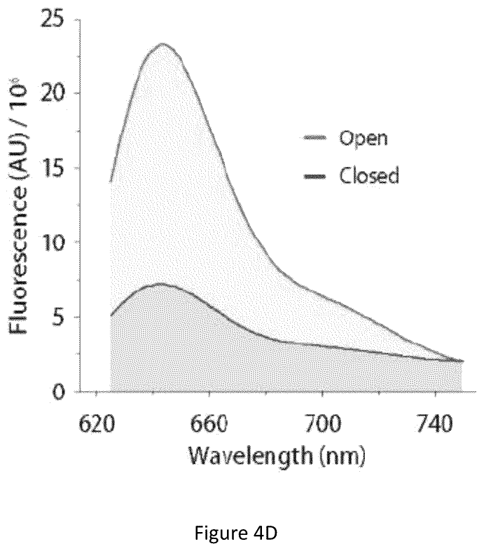

We created two new versions of TraNS, designed to be specific to miR21 and miR23b, that opens and shows fluorescence enhancement upon binding to miR21 (FIG. 4A) and miR23b (FIG. 4D), respectively. We then check the specificity of these two TraNS devices for three NSCLC biomarker miRs, i.e.—miR-21-5p, miR-10b-5p and miR-23b-3p. Compared to threshold fluorescence when no target is present (dashed line—FIG. 4B), TraNS-miR21 showed signal increase only in presence of miR21 but not for miRlOb and miR23b (FIG. 4B). Hence, both the TraNS-miR21 as well as TraNS-miR23b are specific to only their respective target and not toward the wrong targets.

Next, we tested the ability of TraNS-miR21 and TraNS-miR23b to detect their corresponding targets encapsulated inside SUVs. Similar to 1T4Bp-key detection FIG. 2C, we observed signal increase only in case of cholesterol modified TraNS compared to the non-cholesterol analog—TraNS-miR21 (FIG. 4C). This demonstrates that the TraNS device can be applied for membrane enclosed miR sensing.

### Exosomal Micro-RNA Detection by TraNS

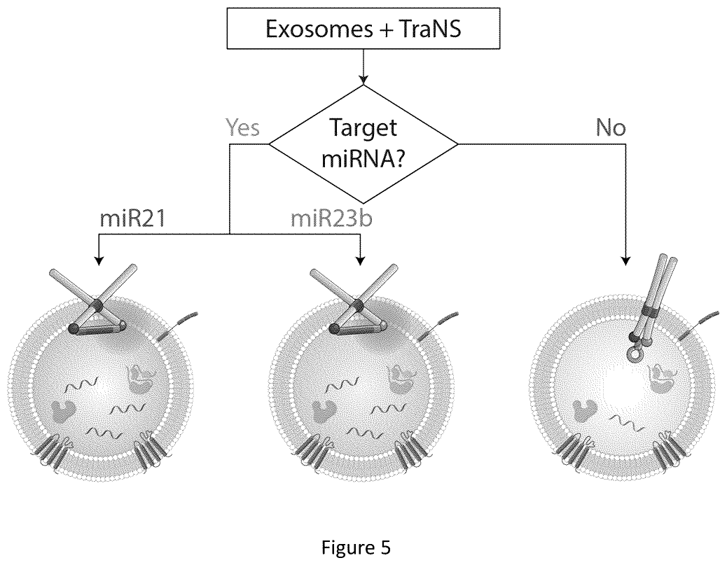

Next, we further expanded the scope of our TraNS device for miR detection in exosomes. The upregulation of the miR21 and miR23b biomarkers in exosomes have previously been associated with poor survival rate of non-small cell lung cancer patients. We used cholesterol modified TraNS-miR21 (FIG. 5—left) and TraNS-miR23b (FIG. 5—middle) to exosomes derived from (i) A549 human non-small cell lung cancer cell lines, and (ii) healthy donor serum (FIG. 5—right). Previous reports have found higher hazard ratio for miR23b compared to miR21 in non-small cell lung cancer⁴².

### Discussion

We have successfully developed a bio-inspired nanomechanical device to mimic the transmembrane signal transduction by GPCR proteins. So far, the structural precision and versatility of DNA nanotechnology has been utilized to construct various biomimetic machineries such as—on channels^{21,23}, lipid scramblases²⁸, membrane sculpting proteins²⁷, molecular motors^{56,57}, DNA helicase⁵⁸ etc. The current report mimics a signal transducing GPCR protein by DNA nanostructure for the first time. The functional performance—judged by the ability to sense membrane enclosed nucleic acid biomarkers without disrupting the membrane—creates a unique opportunity for disease diagnosis and prognosis by sensing exosomal micro-RNA molecules Key to the successful fabrication of the channel were the favorable properties of DNA as a predictable and simple-to-handle building material for de novo design. In addition, the simple architecture of TraNS device was produced with high yields and at low material costs to facilitate the future easy adoption of the technology by other scientists. We envision opportunity for studying nucleic acid signatures in single cell level in a non-invasive way in future using mRNA specific TraNS. Moreover, the structural reconfiguration properties of TraNS can be harnessed for signal amplification outside the membrane for ultrasensitive, low-cost detection. The functionality of TraNS can be reversed to deliver small nucleic acid payloads inside exosome or cells. In conclusion, our report establishes the benefits of using DNA for the construction of advanced artificial signal transduction machineries. It thereby synergistically combines three exciting research areas—DNA nanotechnology, signal transducing GPCR proteins and disease diagnostics—and addresses the demand for functional DNA nanostructures.

### Materials

The internally cholesterol modified DNA oligos were purchased from Sigma Aldrich Inc. with dual HPLC purification. The fluorophore and quencher labelled oligos were purchased from LGC Biosearch Inc. with dual HPLC purification. All other DNA and RNA oligonucleotides were purchased from Integrated DNA Technologies Inc. with standard desalting unless mentioned otherwise. These strands were purified in house with denaturing PAGE gels. DOPC lipid was purchased from Avanti Polar Lipids as chloroform solution. Purified exosomes were purchased from System Biosciences Inc. IANBD-amide was purchased from Thermofisher Scientific.

### Methods

### Transmembrane Nano-Sensor Design and Assembly

The TraNS device nanostructure was designed de novo by Tiamat software⁵⁹. Information on the DNA oligonucleotide sequences, two-dimensional DNA maps are provided in Tables 1 and 2. For assembly of the structure, an equimolar mixture of PAGE purified DNA strands in water was mixed at a final concentration of 1 μM each in a final buffer concentration of 1× lip-DNA buffer (1×TAE, 100 mM KCl, pH 7.4). Concentration of each strand was measured freshly every time before structure assembly, as the correct molar stoichiometry is crucial for final yield. For concentration measurement, each of the strands were diluted 20× in water (1 μL strand+19 μL water), heated at 90° C. for 5-10 mins, centrifuged and mixed well by pipetting up and down before taking 2 μL for A260 measurements in a Thermofisher Nanodrop instrument. The concentration was calculated from the A260 value using the extinction coefficient provided by the manufacturer. The structure was annealed in a Bio-rad PCR thermocycler with heating at 90 ° C. for 5 mins, lowering the temperature by 5 mins for every 2° C. from 90° C. to 76° C. and then by 5 mins for every 4° C. from 76° C. to 24° C. The non-cholesterol version of the nanopores was used for gel and fluorescence spectral characterization for structural reconfiguration.

### Native PAGE

The assembled TraNS devices were analyzed using a 10% polyacrylamide gel using 1×TAE, 12.5mM Mg²⁺ buffer. The gel was run in an Amersham set up (Ruby 600) in buffer chamber containing the same buffer as the gel. 10 pmoles of each sample containing final 1× native loading buffer was loaded in each well. The gel was run at 200V constant voltage (12.5 volt/cm) for 3.5 hours at 20° C. After run, the gel was stained with Ethidium Bromide for 10 mins, followed by destaining with water for 5 mins. The gel was imaged using a Bio-rad gel scanner with Ethidium Bromide filters.

### Preparation of DNA Target-Filled Small Unilamellar Vesicles (SUVs) and Purification of Excess Unencapsulated DNA

The small unilamellar vesicles were synthesized by extrusion method as described elsewhere⁶⁰. Briefly, 3.25 μmoles of DOPC lipid dissolved in chloroform was taken in a 2 mL glass vial and dried under vacuum using a Buchi rotatory evaporator to generate a lipid film. The chloroform was completely removed by keeping the vial at high vacuum for at least 30 mins. The lipid film was suspended in 1× lip-DNA buffer (1×TAE, 100 mM KCl, pH 7.4) by vigorous pipetting and vortexing to generate multilamellar large vesicles (MLVs) at a lipid concentration of 6.5 mM or 5 mg/mL. The MLV solution was subjected to bath sonication for 10 mins, followed by 10 cycles of freezing and thawing. Small unilamellar vesicles (SUVs) were generated by extruding the resulting solution through 0.2 μm polycarbonate membrane followed by 0.1 μm polycarbonate membrane, 22 times through each filter using an Avanti Polar Lipids extrusion set up. For encapsulation of DNA targets, 1× lip-DNA buffer in the rehydration step was replaced by 1× lip-DNA buffer containing desired amount of target DNA or RNA. The excess unencapsulated DNA or RNA target was removed by washing the SUV solution through 15 mL 100 kDa molecular weight cut-off (MWCO) amicon filters. Approximately 0.5-1 mL SUV solution was diluted to final 15 mL by filtered 1× lip-DNA buffer and centrifuged at 2500×g for 30 mins at 4° C. The washing steps were repeated 5 times. The MWCO filter was changed after the 2^{nd} cycle to improve washing efficiency (also as sometimes it got clogged by stuck SUVs).

### SUV Concentration and Size Measurement

The concentration and size distribution of the SUVs were measured by Nanoparticle Tracking Analysis method⁶¹ in Malvern Nanosight NS300 instrument. The stock liposome or exosome solutions were diluted 1000 folds in filtered 1× lip-DNA buffer (1× TAE, 100 mM KCl, pH 7.4, filtered through 100 kDa amicon filter) and mixed well by pipetting up and down for 10 times, vortexing for 30 sec. The sample chamber was first washed with 2 column volumes of the filtered buffer. The screen gain was set to 7, camera level was set to 12 during collection of the tracks. Total 1000-3000 tracks were collected. The tracks were analyzed with the built-in NTA software (NTA 3.4 Build 3.4.003) using screen gain 10-15 and detection threshold of 3-5, to ensure 12-120 particles/frame, suggested for optimal analysis.

### IANBD Labelling

IANBD labelling was performed following a previously published protocol⁵⁵ for labelling iodoacetamide to phosphorothioate (PPT) backbone modified DNA. Briefly—the PPT modified DNA in water was reacted with 100× molar excess of IANBD-amide in 1×PBS. The reaction mixture was incubated at 50° C. with shaking overnight. The excess unreacted IANBD-amide was removed using 3 kDa amicon ultra microspin filters. Finally, the IANBD-DNA was run in a 10% denaturing poly acrylamide gel electrophoresis to purify the construct.

### Fluorescence Spectroscopy

All the fluorescence spectra were acquired in a Nanolog fluorometer (Horiba Jobin Yvon) using a Quartz glass high performance cuvette with 1.5×1.5 mm optical path length from Hellma (article no 105-252-15-40). In atypical experiment 12 μL total solution with 50 nM tweezer and 0.25 nM liposome in 1× lip-DNA buffer was taken. The parameters settings of the fluorimeter for a steady state measurement were as follows: 494 nm excitation (λₘₐₓ^{ex} for FAM dye), 2 nm excitation slit, grating (1200, 330) and emission window between 510-600 nm with an increment of 1 nm, 7 nm emission slit, grating (1200, 500). For kinetics experiments, the emission at 520 nm was monitored from 0 to 4000 sec with a 1 sec interval with 0.5 sec integration time for each signal.

### Preparation of Giant Unilamellar Vesicles (GUVs) for Confocal Measurements

The GUVs were prepared by inverted emulsion method²². POPC (150 μL, 10 mM) in chloroform was added to a 1 mL glass vial, the solvent was removed under vacuum and rotation using a Buchi rotary evaporator set at high vacuum for at least 30 minutes. The thin film generated was resuspended in mineral oil (150 μL) by vortexing and sonicating for 10 minutes. 254 of inner solution (IS, the solution that will be encapsulated inside GUVs) containing ˜435 mOsm/kg sucrose added to the mineral oil. A water-in-oil emulsion was created by suspending the IS into the mineral oil by pipetting up and down for —10 times followed by vortexing at highest speed for ˜30 seconds and sonicating for 10 minutes at room temperature. This emulsion was then carefully added to the top of 1 mL external solution (ES, the solution to be kept outside the GUVs) containing ˜435 mOsm/kg glucose in a plastic microcentrifuge tube. The osmolarities of the IS and ES were measured by an osmometer (Advanced Instruments Model 3320 Osmometer 2996) and balanced properly such that the osmolarity difference is less than 20 mOsm/kg. The GUVs were generated by centrifuging at 21k×g at 4° C. for 15 minutes. The mineral oil top layer and most of the sucrose layer (˜900 μL) was carefully removed by pipettor, every time using a fresh tip. The remaining solution containing the pelleted vesicles was gently mixed with a pipettor, then transferred to a clean plastic vial leaving a small quantity to avoid contamination of remaining trace amount of mineral oil to the GUVs.

### Example 2

### Nanoarray

### Introduction

The current exosome assays characterize ex-BMs in a bulk, analog-based ways via ensemble fluorescence spectra20, surface-plasmon resonance (SPR) or electrochemical methods^{46,47}. Being unable to distinguish the altered abundance of disease-specific exosome sub-population, these assays are non-quantitative and may lead to false diagnostics. The way to circumvent this problem is to profile the exosomal biomarkers in single intact exosomes with high-throughput digital counting. Biomolecules patterned in an array offers more rapid, high-throughput and ultra-sensitive quantification by digital counting of signals compared to traditional analog measurements. Our patented low-cost DNA Origami Nanoarray (DON) offers unique ability to pattern digitally defined number of biomolecules such as DNA, protein, antibody etc. on a glass surface. The DON platform offers a great opportunity to translate our TraNS device based ex-miR sensing to a high-throughput, ultra-sensitive and digitally quantitative assay. In this aim, we propose—1) patterning of intact exosomes isolated from cultured cell line on DON for rapid, high-throughput and ultra-sensitive readout of overall exosome abundance and 2) single molecule digital quantification of target ex-miR-containing exosome subpopulation to assess their role in cancer prognosis.

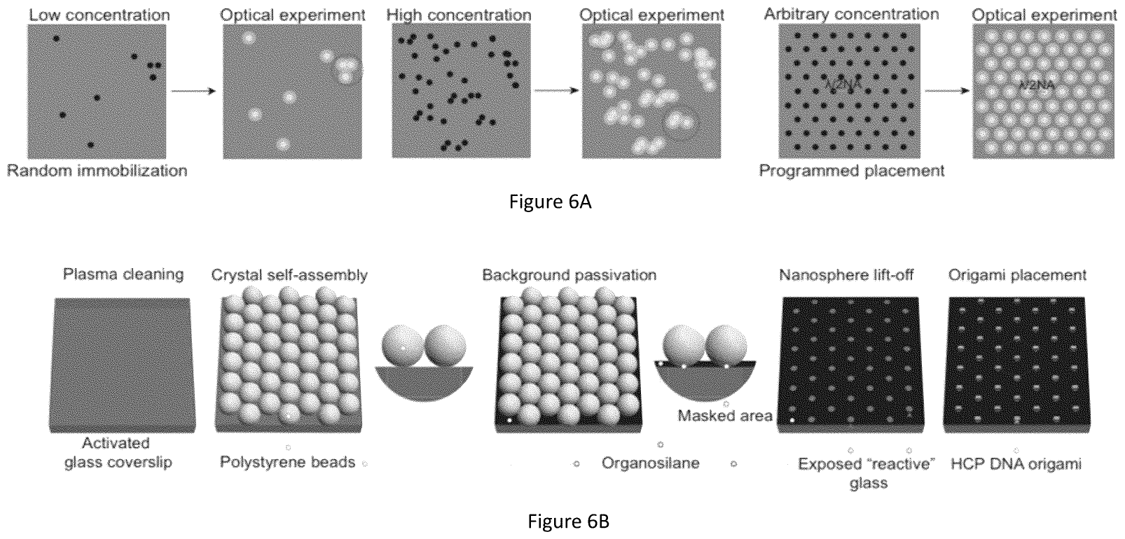

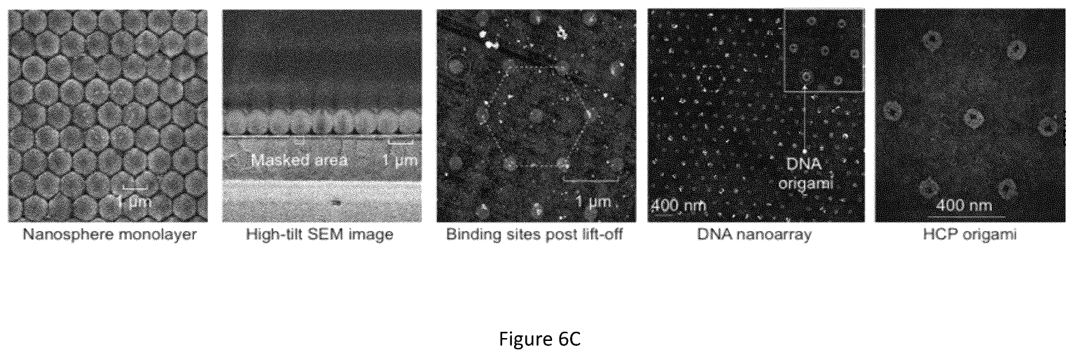

Nanoarray formation. We sought to develop a device platform that would enable single-molecule experiments to be performed at the highest packing density while significantly simplifying the fabrication process (FIG. 6). This meant positioning DNA origami at distinct sites separated by a pitch larger than the diffraction limit of visible light. This overcomes the need to use super-resolution microscopy for resolving individual signaling events occurring on the DNA origami substrate or its payload of functional moieties.

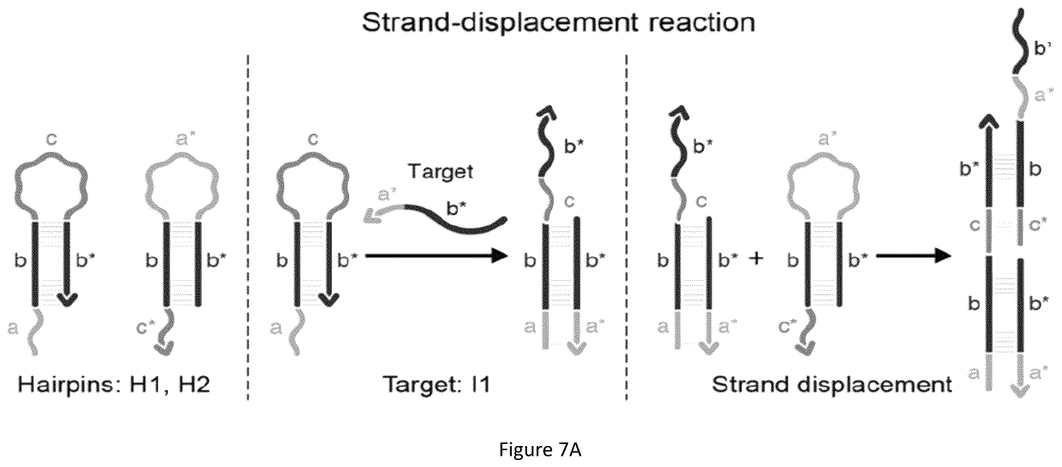

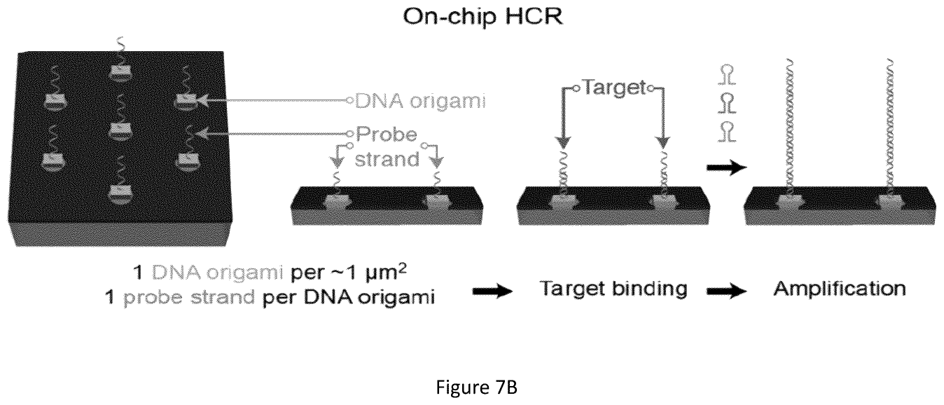

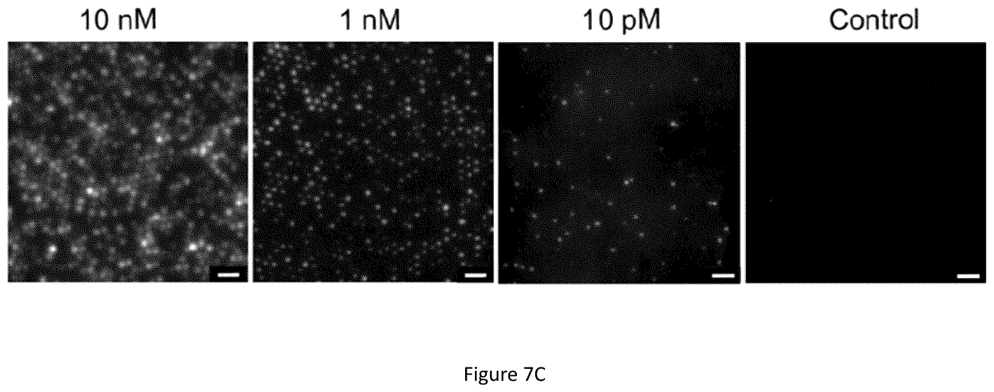

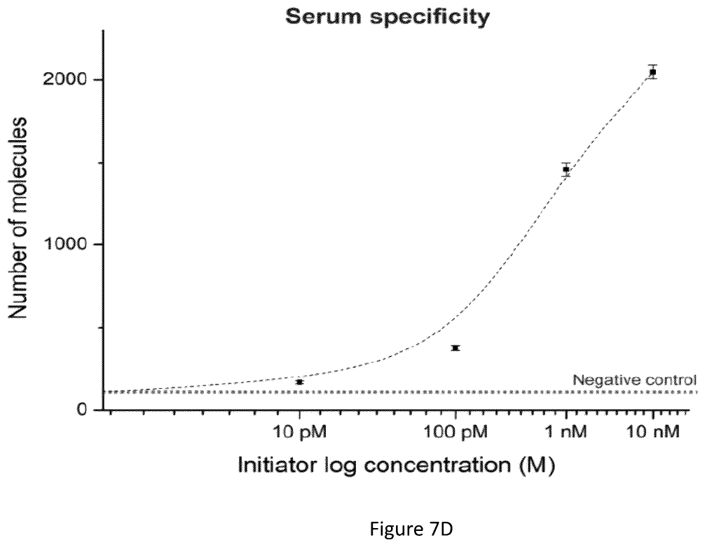

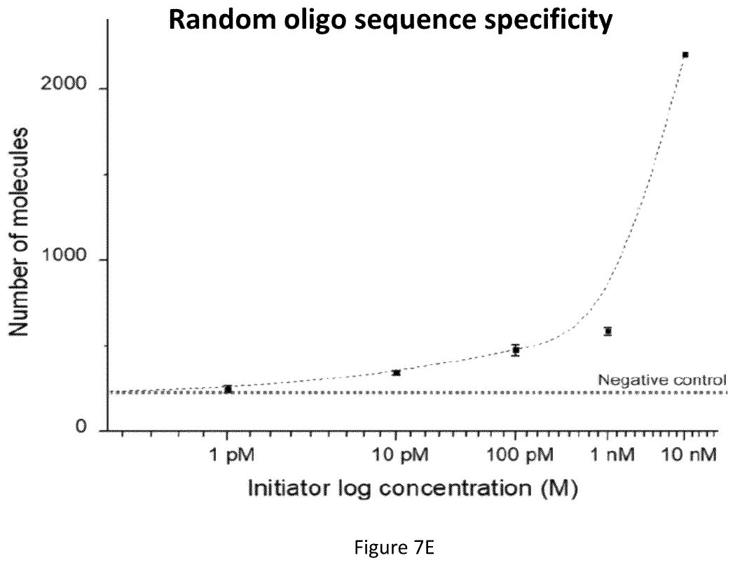

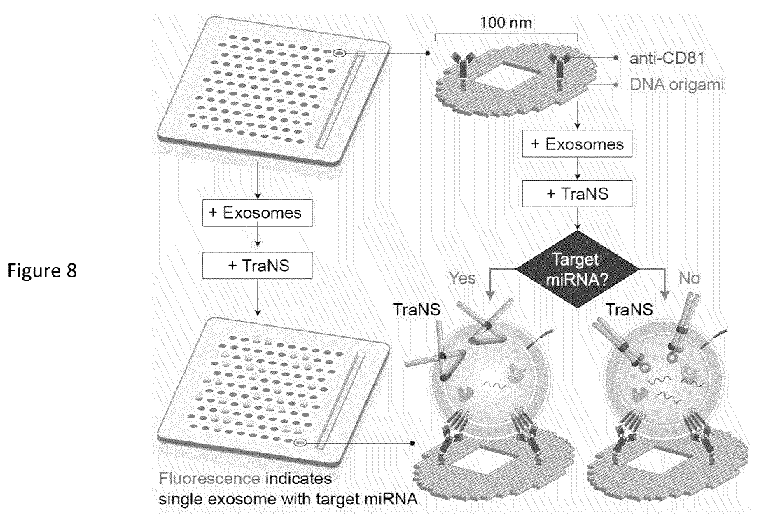

Signal molecule digital quantification. As a proof-of-concept for ultrasensitive and quantitative detection of biomarkers, we have demonstrated a digital assay for the enzyme-free, in situ isothermal hybridization chain reaction (HCR) amplification-based detection of a synthetic ssDNA target (FIG. 7A-B). This is, to our knowledge, the first demonstration of a digital detection technique using DNA origami-bound probe molecules programmatically decorated on a meso-to-macroscale chip (FIG. 7C). The significant advantages of this method are its simplicity, minimal user-engagement, absolute quantification-based detection scheme, enzyme-free amplification, robustness, ˜25 pM limit of detection (FIG. 7D-E), low reagent consumption, turnaround time, and low chip ($1)/assay ($10) cost. We foresee its applications mainly in point-of-care testing (POCT) for detection of all classes of biomolecules, i.e. DNA, RNA, small molecules, proteins by integration with a low-cost, fluorescence microscopy platform.

Capturing and digital quantification of exosomes on DON. Common exosome membrane protein, such as CD81 specific antibody based capture of exosomes on solid surface has been demonstrated previously. We will use biotin-streptavidin bridge to conjugate biotinylated human anti-CD81 antibody to biotinylated DON. Next, isolated exosomes will be bound to the nanoarrays by incubating the exosome solution on DON and washing off unbound exosomes. The formation of the exosome nanoarray will be validated using scanning electron microscopy (SEM). For validation of exosome nanoarray formation by fluorescence microscopy, exosomes will be stained with ExoGlow-Red™ from SBI Inc. The concentration of exosomes will be digitally quantified using fluorescence counting with Mathematica code. We will standardize the experiment using known concentration of exosomes based on NTA measurements.

Quantification of ex-miR based exosome subpopulation. To translate the ex-miR based cancer diagnosis and prognosis for clinical application, the knowledge of biomarker profile in single exosome level will be evaluated. We will use our TraNS technology to quantify the % of exosomes that contain cancer specific ex-miRs. After forming the exosome nanoarray, we will add miR-21-5p or let-7b-3c specific TranS device. The characteristic fluorescence increase will be used to quantify number of exosomes containing target ex-miRs. The % of exosomes containing target ex-miRs will be normalized with respect to total number of exosomes counted from general exosome staining by ExoGlow-Red™.

Data analysis, expected results and interpretation. Anti-CD81 nanoarray formation. The anti-CD81 antibody will be conjugated to the DNA origami using streptavidin bridge between biotinylated DON and biotinylated anti-CD81. This conjugation will be first tested using agarose gel electrophoresis. The band corresponding to the DNA origami will be upshifted upon conjugation to anti-CD81. Formation of the anti-CD81 nanoarray will be characterized by atomic force micrographs (AFM) where we expect to see additional features on each origami in DON. Moreover, the anti-CD81 will be labelled with dye-labelled secondary antibody to visualize using total internal reflection fluorescence microscopy (TIRFM).

Exosome nanoarray formation. Formation of the exosome nanoarray will be characterized by SEM and fluorescence microscopy. Exosomes are easily visible in SEM21, hence we expect to see hexagonally closed packed (HCP) arrangement of exosomes upon their capture on DON. For fluorescence microscopy, we also expect to see hexagonally arranged signals upon imaging using 640 nm LASER in TIRFM.

Exosome quantification. The number of bound exosomes can be easily counted by a manually or using a Mathematica code from the TIRF micrographs. We expect to see a correlation between total exosome concentration (obtained from NTA) and bound exosome concentration (from TIRF B micrographs).

Ex-miR-based exosome subpopulation quantification. Quantitative analysis of the abundance and stoichiometry of ex-miRs in exosomes has not been done extensively in literature. Based on bulk measurements, Chevillet et al have predicted that 1 in every 100 exosomes might have ex-miR content50. DONA-TraNS based digital counting is sensitive enough to quantify this with unprecedented precision. A typical 50 μm×80 μm scan area of TIRFM consists of 57732 binding sites. Even with the predicted 1 in 100 positive events, every snapshot should yield >500 data points. And in a typical 24 mm×20 mm coverslip, approximately 7×10⁷ positive events can be observed in every experiment. Our method will enable first ever digital quantification of ex-miR occupancy and stoichiometry in a single intact exosome level. The knowledge will advance both ex-miR based diagnostics as well as understanding the biogenesis and physiological role of exosomes as carriers of cellular information molecules for cell-cell communication.

### Comments

Non-specific binding of anti-CD81 and/or exosomes to empty spaces of DON. The hydrophobic surface passivating agent hexamethyldisilazane (HMDS) might lead to non-specific binding of anti-CD81 to the surface This problem can be circumvented using blocking agents such as bovine serum albumin (BSA), tween-20, casein etc. in the binding buffer. Alternatively, HMDS layer can be modified to a hydrophilic layer such as PEG-pyrene or PEG-biotin after formation of the nanoarray. Moreover, our noise-filter in the data analysis method is also able to rule out the non-specifically bound spots.

Low binding efficiency of exosomes to anti-CD81-DON. The binding efficiency of the exosomes to the binding spots might be low. The binding efficiency can be optimized by rationally tuning the primary global conditions such as—incubation time, concentration of salt, crowding agents, number of antibodies conjugated to each origami in the DON. Moreover, several exosomal membrane protein (such as EpCAM and EphA2) specific antibodies can be used to improve the binding efficiency of the exosomes.

Multiplexed detection. The disclosed method of digital counting would be very advantageous for multiplexed detection. Co-localization of signal from one exosome upon addition of different ex-miR specific TraNS device with different fluorophores will enable multiplexed detection of ex-miR unprecedented before.

### Example 3

### Rapid, Ultra-Sensitive, Digital Detection and Quantification of ex-miR from Clinical Sample

For clinical translation of ex-miR based liquid biopsy methods, our TraNS device-based ex-miR sensing needs to be tested on clinical samples. Clinical serum samples from pancreatic cancer and healthy patient will be tested. Statistical analysis on the data will be performed to measure differences between cancer and control subjects. The data collected will be evaluated with respect to patient clinical information to further understand if the change in the expression of these two ex-miR markers can help differentiate the different stages of cancer.

TraNS sensor-mediated detection of target ex-miRs in exosomes from clinical serum samples. To test the ability of TraNS device to detect exosomal miR-21-5p and let-7b-3p in clinical sample, we will perform the TraNS-based detection on exosomes from pancreatic cancer patients. As negative control we will use serum from healthy people. The fluorescence-change for cancer vs normal patients will be compared. We will use a logistic regression model to fit our ex-miRs quantification to the clinical data and correspondingly perform the Receiver Operating Characteristic (ROC) curve analysis to determine the sensitivity and specificity of the TraNS device.

Capture and digital quantification of ex-miR containing exosome subpopulation from clinical samples of cancer and normal patients on DON. Exosomes from patient samples will be captured as described above in Example 2. Briefly, serum samples will be incubated with anti-CD81-DON and unbound entities will be washed away. Next, we will quantify the overall number of bound exosomes by staining of the exosomes by ExoGlow-Red™. Finally, the ex-miR containing exosome subpopulation will be quantified by sensing the target ex-miRs inside the captured exosomes using specific TraNS device as described in the case of cancer cell derived exosomes. We will use a logistic regression model to fit our ex-miRs quantification to the clinical data and correspondingly perform the Receiver Operating Characteristic (ROC) curve analysis to determine the sensitivity and specificity of the DON-TraNS assay.

Data analysis, expected results and interpretation. The main advantage of TraNS-based ex-miR detection is to bypass the hassle of exosome isolation, RNA extraction, primer design for qRT-PCR and detection by qRT-PCR. Hence, a sensitivity and specificity closed to the conventional methods will be enough for our purpose at this step. Molecular beacons have previously been used to detect micro-RNA from body fluids. We expect that our molecular beacon-based TraNS devices should have sensitivity and specificity enough for ex-miR detection in clinical samples.

Stability of DNA TraNS devices in blood serum. DNA nanostructures have been shown to be stable in blood serum for >24 hours, which is >10× longer than the expected measurement time. To minimize false positive signal due to DNA degradation, we will employ common strategies for reduce nuclease degradation, such as using modified DNA strands with phosphorthioate backbone and UV crosslinking of DNA strands.

Signal to Noise in fluorescence measurements. The FAM dye used in our preliminary studies has been reported to be prone to photobleaching and strongly dependent on pH of the solution. This might limit the sensitivity of TraNS device for detection from biofluids. If that case, we will use more photostable and robust dyes such as Cy3 or Cy5 and corresponding quencher dye in our TraNS sensor.

### REFERENCES

1 Langton, M. J., Keymeulen, F., Ciaccia, M., Williams, N. H. & Hunter, C. A. Controlled membrane translocation provides a mechanism for signal transduction and amplification. Nat Chem 9, 426-430, doi:10.1038/nchem.2678 (2016).
2 Furge, L. L. Biochemistry of signal transduction and regulation, 4th Ed. by G. Krauss. Biochemistry and Molecular Biology Education 36, 385-385, doi:10.1002/bmb.20226 (2008).
3 Hille, B. Ion channels of excitable membranes. 3rd edn, (Sinauer, 2001).
4 Lemmon, M. A. & Schlessinger, J. Cell Signaling by Receptor Tyrosine Kinases. Cell 141, 1117-1134, doi:10.1016/j.ce11.2010.06.011 (2010).
5 Venkatakrishnan, A. J. et al. Molecular signatures of G-protein-coupled receptors. nature 494, 185-194, doi:10.1038/nature11896 (2013).
6 Sakai, N. & Matile, S. Synthetic Ion Channels. Langmuir 29, 9031-9040, doi:10.1021/1a400716c (2013).
7 Gale, P. A. From Anion Receptors to Transporters. Accounts Chem Res 44, 216-226, doi:10.1021/ar100134p (2011).
8 Bernitzki, K. & Schrader, T. Entirely Artificial Signal Transduction with a Primary Messenger. Angewandte Chemie International Edition 48, 8001-8005, doi:10.1002/anie.200902973 (2009).
9 Barton, P., Hunter, C. A., Potter, T. J., Webb, S. J. & Williams, N. H. Transmembrane Signalling. Angewandte Chemie International Edition 41, 3878-3881, doi:10.1002/1521-3773(20021018)41:20<3878::Aid-anie3878>3.0.Co;24 (2002).
10 Kasianowicz, J. J., Brandin, E., Branton, D. & Deamer, D. W. Characterization of individual polynucleotide molecules using a membrane channel. Proceedings of the National Academy of Sciences 93, 13770-13773, doi:10.1073/pnas.93.24.13770 (1996).
11 Movileanu, L. Interrogating single proteins through nanopores: challenges and opportunities. Trends in Biotechnology 27, 333-341, doi: 10.1016/j .tibtech.2009. 02.008 (2009).
12 Cheng, Z. et al. Luciferase Reporter Assay System for Deciphering GPCR Pathways. Current Chemical Genomics 4, 84-91, doi:10.2174/1875397301004010084 (2010).
13 Dupin, A. & Simmel, F. C. Signalling and differentiation in emulsion-based multi-compartmentalized in vitro gene circuits. Nat Chem 11, 32-39, doi:10.1038/s41557-018-0174-9 (2018).
14 Villar, G., Graham, A. D. & Bayley, H. A Tissue-Like Printed Material. Science 340, 48-52, doi:10.1126/science.1229495 (2013).
15 Manglik, A. & Kobilka, B. The role of protein dynamics in GPCR function: insights from the beta2AR and rhodopsin. Curr Opin Cell Biol 27, 136-143, doi:10.1016/j.ceb.2014.01.008 (2014).
16 Langton, M. J., Keymeulen, F., Ciaccia, M., Williams, N. H. & Hunter, C. A. Controlled membrane translocation provides a mechanism for signal transduction and amplification. Nat Chem 9, 426-430, doi:10.1038/nchem.2678 (2017).
17 Lister, F. G. A., Le Bailly, B. A. F., Webb, S. J. & Clayden, J. Ligand-modulated conformational switching in a fully synthetic membrane-bound receptor. Nat Chem 9, 420-425, doi:10.1038/Nchem.2736 (2017).
18 Chen, J. & Seeman, N. C. Synthesis from DNA of a molecule with the connectivity of a cube. Nature 350, 631-633, doi:10.1038/350631a0 (1991).
19 Rothemund, P. W. K. Folding DNA to create nanoscale shapes and patterns. Nature 440, 297-302, doi:10.1038/nature04586 (2006).
20 Douglas, S. M. et al. Self-assembly of DNA into nanoscale three-dimensional shapes. Nature 459, 414-418, doi:10.1038/nature08016 (2009).
21 Burns, J. R., Seifert, A., Fertig, N. & Howorka, S. A biomimetic DNA-based channel for the ligand-controlled transport of charged molecular cargo across a biological membrane. Nat Nanotechnol 11, 152-156, doi:10.1038/nnano.2015.279 (2016).
22 Krishnan, S. et al. Molecular transport through large-diameter DNA nanopores. Nat Commun 7, 12787, doi:10.1038/ncomms12787 (2016).
23 Langecker, M. et al. Synthetic Lipid Membrane Channels Formed by Designed DNA Nanostructures. Science 338, 932-936, doi:10.1126/science.1225624 (2012).
24 Gopfrich, K. et al. Ion Channels Made from a Single Membrane-Spanning DNA Duplex. Nano Letters 16, 4665-4669, doi:10.1021/acs.nanolett.6b02039 (2016).
25 Thomsen, R. P. et al. A large size-selective DNA nanopore with sensing applications. Nature Communications 10, doi:10.1038/s41467-019-13284-1 (2019).
26 Diederichs, T. et al. Synthetic protein-conductive membrane nanopores built with DNA. Nature Communications 10, doi:10.1038/s41467-019-12639-y (2019).
27 List, J., Weber, M. & Simmel, F. C. Hydrophobic Actuation of a DNA Origami Bilayer Structure. Angewandte Chemie International Edition 53, 4236-4239, doi:10.1002/anie.201310259 (2014).
28 Ohmann, A. et al. A synthetic enzyme built from DNA flips 107 lipids per second in biological membranes. Nature Communications 9, doi:10.1038/s41467-018-04821-5 (2018).
29 Walter, H. -K. et al. “DNA Origami Traffic Lights” with a Split Aptamer Sensor for a Bicolor Fluorescence Readout. Nano Letters 17, 2467-2472, doi:10.1021/acs.nanolett.7b00159 (2017).
30 Kuzyk, A. et al. Reconfigurable 3D plasmonic metamolecules. Nature Materials 13, 862-866, doi:10.1038/nmat4031 (2014).
31 Liu, M. et al. A DNA tweezer-actuated enzyme nanoreactor. Nature Communications 4, doi:10.1038/ncomms3127 (2013).
32 Funke, J. J. & Dietz, H. Placing molecules with Bohr radius resolution using DNA origami. Nat Nanotechnol 11, 47-52, doi:10.1038/nnano.2015.240 (2015).
33 Zhou, C., Yang, Z. & Liu, D. Reversible Regulation of Protein Binding Affinity by a DNA Machine. Journal of the American Chemical Society 134, 1416-1418, doi:10.1021/ja209590u (2012).
34 Wan, M., Ning, B., Spiegel, S., Lyon, C. J. & Hu, T. Y. Tumor-derived exosomes (TDEs): How to avoid the sting in the tail. Medicinal Research Reviews 40, 385-412, doi:10.1002/med.21623 (2019).
35 H. Rashed, M. et al. Exosomes: From Garbage Bins to Promising Therapeutic Targets. International Journal of Molecular Sciences 18, doi:10.3390/ijms18030538 (2017).
36 Hessvik, N. P., Sandvig, K. & Llorente, A. Exosomal miRNAs as Biomarkers for Prostate Cancer. Frontiers in Genetics 4, doi:10.3389/fgene.2013.00036 (2013).
37 Bhome, R. et al. Exosomal microRNAs (exomiRs): Small molecules with a big role in cancer. Cancer Letters 420, 228-235, doi:10.1016/j.canlet.2018.02.002 (2018).
38 Li, X. et al. Challenges and opportunities in exosome research—Perspectives from biology, engineering, and cancer therapy. APL Bioengineering 3, doi:10.1063/1.5087122 (2019).
39 Morales-Kastresana, A. et al. Labeling Extracellular Vesicles for Nanoscale Flow Cytometry. Scientific Reports 7, doi:10.1038/s41598-017-01731-2 (2017).
40 Willis, G. R., Kourembanas, S. & Mitsialis, S. A. Toward Exosome-Based Therapeutics: Isolation, Heterogeneity, and Fit-for-Purpose Potency. Frontiers in Cardiovascular Medicine 4, doi: 10.3389/fcvm.2017. 00063 (2017).
41 Moriyama, T. et al. MicroRNA-21 modulates biological functions of pancreatic cancer cells including their proliferation, invasion, and chemoresistance. Molecular Cancer Therapeutics 8, 1067-1074, doi:10.1158/1535-7163.Mct-08-0592 (2009).
42 Liu, Q. et al. Circulating exosomal microRNAs as prognostic biomarkers for non-small-cell lung cancer. Oncotarget 8, doi:10.18632/oncotarget.14369 (2016).
43 Zheng, W. et al. MicroRNA-21: A promising biomarker for the prognosis and diagnosis of non-small cell lung cancer (Review). Oncology Letters, doi:10.3892/ol.2018.8972 (2018).
44 Liu, Y. & West, S. C. Happy Hollidays: 40th anniversary of the Holliday junction. Nature Reviews Molecular Cell Biology 5, 937-944, doi:10.1038/nrm1502 (2004).
45 Holliday, R. A mechanism for gene conversion in fungi. Genetical Research 5, 282-304, doi:10.1017/s0016672300001233 (2009).
46 Dhakal, S. et al. Rational design of DNA-actuated enzyme nanoreactors guided by single molecule analysis. Nanoscale 8, 3125-3137, doi:10.1039/c5nr07263h (2016).
47 Tyagi, S. & Kramer, F. R. Molecular Beacons: Probes that Fluoresce upon Hybridization. Nat Biotechnol 14, 303-308, doi:10.1038/nbt0396-303 (1996).
49 Arnott, P. M. & Howorka, S. A Temperature-Gated Nanovalve Self-Assembled from DNA to Control Molecular Transport across Membranes. ACS Nano 13, 3334-3340, doi:10.1021/acsnano.8b09200 (2019).
50 Lanphere, C., Arnott, P. M., Jones, S. F., Korlova, K. & Howorka, S. A Biomimetic DNA-Based Membrane Gate for Protein-Controlled Transport of Cytotoxic Drugs. Angewandte Chemie International Edition, doi:10.1002/anie.202011583 (2020).
51 Agafonov, A. et al. A permeability transition in liposomes induced by the formation of Ca2+/palmitic acid complexes. Biochimica et Biophysica Acta (BBA)—Biomembranes 1609, 153-160, doi:10.1016/s0005-2736(02)00666-1 (2003).
52 Galloux, M. et al. Membrane Interaction of Botulinum Neurotoxin A Translocation (T) Domain. Journal of Biological Chemistry 283, 27668-27676, doi:10.1074/jbc.M802557200 (2008).
53 Shepard, L. A. et al. Identification of a Membrane-Spanning Domain of the Thiol-Activated Pore-Forming ToxinClostridium perfringensPerfringolysin O: An α-Helical to β-Sheet Transition Identified by Fluorescence Spectroscopy†. Biochemistry 37, 14563-14574, doi:10.1021/bi981452f (1998).
54 Mesmin, B. et al. Two Lipid-Packing Sensor Motifs Contribute to the Sensitivity of ArfGAP1 to Membrane Curvature†. Biochemistry 46, 1779-1790, doi:10.1021/bi062288w (2007).
55 Lee, J. H. et al. Site-Specific Control of Distances between Gold Nanoparticles Using Phosphorothioate Anchors on DNA and a Short Bifunctional Molecular Fastener. Angewandte Chemie International Edition 46, 9006-9010, doi:10.1002/anie.200702569 (2007).
56 Omabegho, T., Sha, R. & Seeman, N. C. A bipedal DNA Brownian motor with coordinated legs. Science 324, 67-71, doi:10.1126/science.1170336 (2009).
57 Hariadi, R. F. et al. Mechanical coordination in motor ensembles revealed using engineered artificial myosin filaments. Nat Nanotechnol 10, 696-700, doi:10.1038/Nnano.2015.132 (2015).
58 Kosuri, P., Altheimer, B. D., Dai, M., Yin, P. & Zhuang, X. Rotation tracking of genome-processing enzymes using DNA origami rotors. Nature 572, 136-140, doi:10.1038/s41586-019-1397-7 (2019).
59 Williams, S. et al. in DNA Computing Lecture Notes in Computer Science Ch. Chapter 8, 90-101 (2009).
60 Hope, M. J., Bally, M. B., Webb, G. & Cullis, P. R. Production of large unilamellar vesicles by a rapid extrusion procedure. Characterization of size distribution, trapped volume and ability to maintain a membrane potential. Biochimica et Biophysica Acta (BBA)—Biomembranes 812, 55-65, doi:10.1016/0005-2736(85)90521-8 (1985).
61 Vestad, B. et al. Size and concentration analyses of extracellular vesicles by nanoparticle tracking analysis: a variation study. Journal of Extracellular Vesicles 6, 1344087, doi:10.1080/20013078.2017.1344087 (2017).
62 Vaidyanathan, R., Soon, R. H., Zhang, P., Jiang, K. & Lim, C. T. Cancer Diagnosis: From Tumor to Liquid Biopsy and Beyond. Lab on a Chip, doi:10.1039/c81c00684a (2018).
63 Eccles, S. A. & Welch, D. R. Metastasis: recent discoveries and novel treatment strategies. The Lancet 369, 1742-1757, doi:10.1016/s0140-6736(07)60781-8 (2007).
64 Nagrath, S. et al. Isolation of rare circulating tumour cells in cancer patients by microchip technology. Nature 450, 1235-1239, doi:10.1038/nature06385 (2007).
65 Diehl, F. et al. Circulating mutant DNA to assess tumor dynamics. Nature Medicine 14, 985-990, doi:10.1038/nm.1789 (2008).
66 Danila, D. C. et al. Circulating Tumor Cell Number and Prognosis in Progressive Castration-Resistant Prostate Cancer. Clin Cancer Res 13, 7053-7058, doi:10.1158/1078-0432.Ccr-07-1506 (2007).
67 Gormally, E. et al. Amount of DNA in plasma and cancer risk: A prospective study. International Journal of Cancer 111, 746-749, doi:10.1002/ijc.20327 (2004).
68 de Vrij, J. et al. Quantification of nanosized extracellular membrane vesicles with scanning ion occlusion sensing. Nanomedicine 8, 1443-1458, doi:10.2217/nnm.12.173 (2013).
69 Crawley, A. S. & O'Kennedy, R. J. The need for effective pancreatic cancer detection and management: a biomarker-based strategy. Expert Review of Molecular Diagnostics 15, 1339-1353, doi:10.1586/14737159.2015.1083862 (2015).
70 Nuzhat, Z. et al. Tumour-derived exosomes as a signature of pancreatic cancer - liquid biopsies as indicators of tumour progression. Oncotarget 8, doi:10.18632/oncotarget.13973 (2016).
71 Li, Y. & Sarkar, F. H. MicroRNA Targeted Therapeutic Approach for Pancreatic Cancer. International Journal of Biological Sciences 12, 326-337, doi:10.7150/ijbs.15017 (2016).
72 Kuwabara, Y. et al. Increased MicroRNA-1 and MicroRNA-133a Levels in Serum of Patients With Cardiovascular Disease Indicate Myocardial Damage. Circulation: Cardiovascular Genetics 4, 446-454, doi:10.1161/circgenetics.110.958975 (2011).
73 Théry, C., Amigorena, S., Raposo, G. & Clayton, A. Isolation and Characterization of Exosomes from Cell Culture Supernatants and Biological Fluids. Current Protocols in Cell Biology 30, 3.22.21-23.22.29, doi:10.1002/0471143030.cb0322s30 (2006).
74 Dragovic, R. A. et al. Sizing and phenotyping of cellular vesicles using Nanoparticle Tracking Analysis. Nanomedicine: Nanotechnology, Biology and Medicine 7, 780-788, doi:10.1016/j.nano.2011.04.003 (2011).
75 Pinheiro, A. V., Han, D., Shih, W. M. & Yan, H. Challenges and opportunities for structural DNA nanotechnology. Nat Nanotechnol 6, 763-772, doi:10.1038/nnano.2011.187 (2011).
76 Baker, M. B., Bao, G. & Searles, C. D. In vitro quantification of specific microRNA using molecular beacons. Nucleic Acids Research 40, e13-e13, doi:10.1093/nar/gkr1016 (2012).
77 Choi, H. M. T. et al. Third-generationin situhybridization chain reaction: multiplexed, quantitative, sensitive, versatile, robust. Development 145, doi:10.1242/dev.165753 (2018).
78 Daems, D. et al. Three-Dimensional DNA Origami as Programmable Anchoring Points for Bioreceptors in Fiber Optic Surface Plasmon Resonance Biosensing. ACS Applied Materials & Interfaces 10, 23539-23547, doi:10.1021/acsami.8b04757 (2018).
79 Bi, S., Chen, M., Jia, X., Dong, Y. & Wang, Z. Hyperbranched Hybridization Chain Reaction for Triggered Signal Amplification and Concatenated Logic Circuits. Angewandte Chemie International Edition 54, 8144-8148, doi:10.1002/anie.201501457 (2015).
80 Bao, G., Rhee, W. J. & Tsourkas, A. Fluorescent Probes for Live-Cell RNA Detection. Annual Review of Biomedical Engineering 11, 25-47, doi:10.1146/annurev-bioeng-061008-124920 (2009).
81 Lee, J., Kwon, M. H., Kim, J. A. & Rhee, W. J. Detection of exosome miRNAs using molecular beacons for diagnosing prostate cancer. Artificial Cells, Nanomedicine, and Biotechnology 46, S52-S63, doi:10.1080/21691401.2018.1489263 (2018).
82 Yang, Y. et al. An Immuno-Biochip Selectively Captures Tumor-Derived Exosomes and Detects Exosomal RNAs for Cancer Diagnosis. ACS Applied Materials & Interfaces 10, 43375-43386, doi:10.1021/acsami.8b13971 (2018).
83 Burns, J. R., Seifert, A., Fertig, N. & Howorka, S. A biomimetic DNA-based channel for the ligand-controlled transport of charged molecular cargo across a biological membrane. Nat Nanotechnol 11, 152-156, doi:10.1038/nnano.2015.279 (2016).
84 Krishnan, S. et al. Molecular transport through large-diameter DNA nanopores. Nat Commun 7, 12787, doi:10.1038/ncomms12787 (2016).
85 Langecker, M., Arnaut, V., List, J. & Simmel, F. C. DNA Nanostructures Interacting with Lipid Bilayer Membranes. Accounts Chem Res 47, 1807-1815, doi:10.1021/ar500051r (2014).
86 Chen, H. & Li, J. Nanotechnology: moving from microarrays toward nanoarrays. Methods Mol Biol 381, 411-436, doi:10.1007/978-1-59745-303-5 22 (2007).
87 Basu, A. S. Digital Assays Part II: Digital Protein and Cell Assays. SLAS TECHNOLOGY: Translating Life Sciences Innovation 22, 387-405, doi:10.1177/2472630317705681 (2017).
88 Gopinath, A., Rothemund, P., Shetty, R. & Hariadi, R. Materials And Methods Relating To Single Molecule Arrays. WO patent WO 2019/108954 Al (2019).
89 Georges, J. F. et al. Use of a Conformational Switching Aptamer for Rapid and Specific Ex Vivo Identification of Central Nervous System Lymphoma in a Xenograft Model. Plos One 10, doi:ARTN e0123607 10.1371/journal.pone.0123607 (2015).
90 Jiang, D. W. et al. DNA origami nanostructures can exhibit preferential renal uptake and alleviate acute kidney injury. Nat Biomed Eng 2, 865-877, doi:10.1038/s41551-018-0317-8 (2018).
91 Li, S. P. et al. A DNA nanorobot functions as a cancer therapeutic in response to a molecular trigger in vivo. Nat Biotechnol 36, 258-+, doi:10.1038/nbt.4071 (2018).
92 Hariadi, R. F., Cale, M. & Sivaramakrishnan, S. Myosin lever arm directs collective motion on cellular actin network. P Natl Acad Sci USA 111, 4091-4096, doi:10.1073/pnas.1315923111 (2014).
93 Hariadi, R. F. et al. Mechanical coordination in motor ensembles revealed using engineered artificial myosin filaments. Nat Nanotechnol 10, 696-700, doi:10.1038/Nnano.2015.132 (2015).
94 Bapat, A. A., Munoz, R. M., Von Hoff, D. D. & Han, H. Blocking Nerve Growth Factor Signaling Reduces the Neural Invasion Potential of Pancreatic Cancer Cells. Plos One 11, e0165586, doi:10.1371/journal.pone.0165586 (2016).
95 Whatcott, C. J. et al. Desmoplasia in Primary Tumors and Metastatic Lesions of Pancreatic Cancer. Clin Cancer Res 21, 3561-3568, doi:10.1158/1078-0432.Ccr-14-1051 (2015).
96 Whatcott, C. J. et al. Inhibition of ROCK1 kinase modulates both tumor cells and stromal fibroblasts in pancreatic cancer. Plos One 12, doi:ARTN e0183871 10.1371/journal.pone.0183871 (2017).
97 Bao, B. et al. Metformin Inhibits Cell Proliferation, Migration and Invasion by Attenuating CSC Function Mediated by Deregulating miRNAs in Pancreatic Cancer Cells. Cancer Prevention Research 5, 355-364, doi:10.1158/1940-6207.Capr-11-0299 (2011).
98 Rodrigues, M., Richards, N., Ning, B., Lyon, C. J. & Hu, T. Y. Rapid Lipid-Based Approach for Normalization of Quantum-Dot-Detected Biomarker Expression on Extracellular Vesicles in Complex Biological Samples. Nano Letters, doi: 10.1021/acs.nanolett.9b02232 (2019).
99 Zhang, W. et al. Characterization of exosomes derived from ovarian cancer cells and normal ovarian epithelial cells by nanoparticle tracking analysis. Tumor Biology 37, 4213-4221, doi:10.1007/s13277-015-4105-8 (2016).
100 Vestad, B. et al. Size and concentration analyses of extracellular vesicles by nanoparticle tracking analysis: a variation study. Journal of Extracellular Vesicles 6, 1344087, doi:10.1080/20013078.2017.1344087 (2017).
101 Liu, T. et al. EVmiRNA: a database of miRNA profiling in extracellular vesicles. Nucleic Acids Res 47, D89-D93, doi:10.1093/nar/gky985 (2019).
102 Luo, L. et al. A ratiometric electrochemical DNA biosensor for detection of exosomal MicroRNA. Talanta 207, doi:10.1016/j.talanta.2019.120298 (2020).
103 Ramshani, Z. et al. Extracellular vesicle microRNA quantification from plasma using an integrated microfluidic device. Communications Biology 2, doi:10.1038/s42003-019-0435-1 (2019).
104 Konry, T., Hayman, R. B. & Walt, D. R. Microsphere-Based Rolling Circle Amplification Microarray for the Detection of DNA and Proteins in a Single Assay. Anal Chem 81, 5777-5782, doi:10.1021/ac900694y (2009).
105 Kowal, J. et al. Proteomic comparison defines novel markers to characterize heterogeneous populations of extracellular vesicle subtypes. Proceedings of the National Academy of Sciences 113, E968-E977, doi:10.1073/pnas.1521230113 (2016).
106 Chevillet, J. R. et al. Quantitative and stoichiometric analysis of the microRNA content of exosomes. Proceedings of the National Academy of Sciences 111, 14888-14893, doi:10.1073/pnas.1408301111 (2014).
107 Lee, J. H., Kim, J. A., Jeong, S. & Rhee, W. J. Simultaneous and multiplexed detection of exosome microRNAs using molecular beacons. Biosensors and Bioelectronics 86, 202-210, doi:10.1016/j.bios.2016.06.058 (2016).
108 Lee, J. H., Kim, J. A., Kwon, M. H., Kang, J. Y. & Rhee, W. J. In situ single step detection of exosome microRNA using molecular beacon. Biomaterials 54, 116-125, doi:10.1016/j.biomaterials.2015.03.014 (2015).

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A transmembrane nanosensor device comprising a lipid conjugated DNA tweezer comprising a hairpin loop complementary to a target polynucleotide trigger strand;
a fluorophore and a quencher paired to the fluorophore, or a FRET pair;
wherein when the hairpin loop is bound by the target polynucleotide trigger strand, the DNA tweezer transitions from a closed conformation to an open conformation, the quencher is separated from the fluorophore, and the fluorophore fluoresces.

2. The transmembrane nanosensor device of claim 1, wherein the lipid conjugated DNA tweezer is integrated into a lipid bilayer.

3. The transmembrane nanosensor of claim 2, wherein the lipid bilayer is an exosome membrane.

4. The transmembrane nanosensor of claim 1, wherein the target polynucleotide trigger strand is an RNA or a DNA polynucleotide.

5. The transmembrane nanosensor of claim 4, wherein the target polynucleotide trigger strand is a micro RNA (miRNA).

6. The transmembrane nanosensor of claim 1, wherein the lipid is a cholesterol molecule.

7. The transmembrane nanosensor of claim 1, comprising one or more of SEQ ID NOs: 1-26.

8. The transmembrane nanosensor of claim 1, wherein the hairpin loop is selected from the group consisting of SEQ ID NO: 31-34.

9. The transmembrane nanosensor of claim 1, comprising an initiator sequence.

10. A method of diagnosing a disease in a subject comprising
contacting exosomes or cells from a subject with the transmembrane nanosensor of claim 1; and
measuring fluorescence of the transmembrane nanosensor, wherein fluorescence of the nanosensor indicates the presence of the disease in the subject.

11. The method of claim 10, wherein the exosomes or cells are from a liquid biological sample from the subject.

12. The method of claim 10, wherein the target polynucleotide trigger is an miRNA biomarker specific to cancer.

13. The method of claim 10, wherein the quantity of fluorescence is measured.

14. An exosomal nanoarray comprising
exosomes bound to a solid surface; and
the transmembrane nanosensor of claim 1.

15. The exosomal nanoarray of claim 14, wherein the exosomes are bound to the solid surface by an exosome specific antibody.

16. The exosomal nanoarray of claim 15, wherein the exosome specific antibody is anti-CD81.

17. The exosomal nanoarray of claim 14, wherein the exosomes are from a patient sample.

18. The exosomal nanoarray of claim 17, wherein the patient sample is a liquid biological sample.


SEQUENCE LISTING (35 sequences)

SEQ ID NO: 1 - DNA, 74 bp, synthetic construct
  misc_feature 1..74: note = Synthetic- sequence 1 for TraNS device
  source 1..74: mol_type = other DNA
tttttggcccgccgcagtatacaacctgaccaataacaacgcggagagcgggtacatgcctggtccacgttttt

SEQ ID NO: 2 - DNA, 85 bp, synthetic construct
  misc_feature 1..85: note = Synthetic- sequence 2 for TraNS device
  source 1..85: mol_type = other DNA
cgtggaccaggcatgtacccgctctccgcgttgttattggtcaccggttgtggagccgcggttcgtaccttcggtgtagctcggg

SEQ ID NO: 3 - DNA, 73 bp, synthetic construct
  misc_feature 1..73: note = Synthetic- sequence 4 for TraNS device
  source 1..73: mol_type = other DNA
tttttcccgagctacaccgaaggtacgaaccgcggctccacaaccggacacaatcgatttttcggtcgttttt

SEQ ID NO: 4 - DNA, 65 bp, synthetic construct
  misc_feature 1..65: note = Synthetic- 3-key sequence for TraNS device
  modified_base 37: mod_base = OTHER; note = BHQ-1 quencher
  modified_base 49: mod_base = OTHER; note = FAM fluorophore
  source 1..65: mol_type = other DNA
tcgattgtgtggttgtatactgcggcgggcctgcggtaagacccacaatccgctcgaccgaaaaa

SEQ ID NO: 5 - DNA, 65 bp, synthetic construct; identical to SEQ ID NO: 4
  misc_feature 1..65: note = Synthetic- 3-key without fluorophores for TraNS device
  source 1..65: mol_type = other DNA

SEQ ID NO: 6 - DNA, 64 bp, synthetic construct
  misc_feature 1..64: note = Synthetic- 3-miR21 sequence for TraNS device
  modified_base 38: mod_base = OTHER; note = FAM fluorophore
  modified_base 47: mod_base = OTHER; note = BHQ-1 quencher
  source 1..64: mol_type = other DNA
tcgattgtgtggttgtatactgcggcgggcctcagactcaacatcatgtctgacgaccgaaaaa

SEQ ID NO: 7 - DNA, 72 bp, synthetic construct
  misc_feature 1..72: note = Synthetic- miR23b sequence for TraNS device
  modified_base 35: mod_base = OTHER; note = BHQ-2 quencher
  modified_base 57: mod_base = OTHER; note = CAL Fluor Red 635 fluorophore
  source 1..72: mol_type = other DNA
tcgattgtgtggttgtatactgcggcgggcctggtaatccctggcaatgtgatgattacctcgaccgaaaaa

SEQ ID NO: 8 - DNA, 74 bp, synthetic construct; identical to SEQ ID NO: 1
  misc_feature 1..74: note = Synthetic- 1-Chol1 sequence for TraNS device
  modified_base 27: mod_base = OTHER; note = Cholesterol TEG
  source 1..74: mol_type = other DNA

SEQ ID NO: 9 - DNA, 74 bp, synthetic construct; identical to SEQ ID NO: 1
  misc_feature 1..74: note = Synthetic- 1-Chol2 sequence for TraNS device
  modified_base 24: mod_base = OTHER; note = Cholesterol TEG
  modified_base 27: mod_base = OTHER; note = Cholesterol TEG
  source 1..74: mol_type = other DNA

SEQ ID NO: 10 - DNA, 73 bp, synthetic construct; identical to SEQ ID NO: 3
  misc_feature 1..73: note = Synthetic- 4-Chol1 sequence for TraNS device
  modified_base 46: mod_base = OTHER; note = Cholesterol TEG
  source 1..73: mol_type = other DNA

SEQ ID NO: 11 - DNA, 73 bp, synthetic construct; identical to SEQ ID NO: 3
  misc_feature 1..73: note = Synthetic- 4-Chol2 sequence for TraNS device
  modified_base 46: mod_base = OTHER; note = Cholesterol TEG
  modified_base 49: mod_base = OTHER; note = Cholesterol TEG
  source 1..73: mol_type = other DNA

SEQ ID NO: 12 - DNA, 23 bp, synthetic construct
  misc_feature 1..23: note = Synthetic- 1T4Bp-key sequence for TraNS device
  source 1..23: mol_type = other DNA
agcggattgtgggtcttaccgca

SEQ ID NO: 13 - DNA, 22 bp, synthetic construct
  misc_feature 1..22: note = Synthetic- miR21-5p sequence for TraNS device
  source 1..22: mol_type = other DNA
tagcttatcagactgatgttga

SEQ ID NO: 14 - DNA, 21 bp, synthetic construct
  misc_feature 1..21: note = Synthetic- miR23b-3p sequence for TraNS device
  source 1..21: mol_type = other DNA
atcacattgccagggattacc

SEQ ID NO: 15 - DNA, 85 bp, synthetic construct; identical to SEQ ID NO: 2
  misc_feature 1..85: note = Synthetic- 2L1 sequence for TraNS device
  modified_base 1: mod_base = OTHER; note = IANBD modification
  source 1..85: mol_type = other DNA

SEQ ID NO: 16 - DNA, 85 bp, synthetic construct; identical to SEQ ID NO: 2
  misc_feature 1..85: note = Synthetic- 2-L34 sequence for TraNS device
  modified_base 34: mod_base = OTHER; note = IANBD modification
  source 1..85: mol_type = other DNA

SEQ ID NO: 17 - DNA, 85 bp, synthetic construct; identical to SEQ ID NO: 2
  misc_feature 1..85: note = Synthetic- 2-L53 sequence for TraNS device
  modified_base 53: mod_base = OTHER; note = IANBD modification
  source 1..85: mol_type = other DNA

SEQ ID NO: 18 - DNA, 85 bp, synthetic construct; identical to SEQ ID NO: 2
  misc_feature 1..85: note = Synthetic- 2-L50 sequence for TraNS device
  modified_base 37: mod_base = OTHER; note = IANBD modification
  source 1..85: mol_type = other DNA

SEQ ID NO: 19 - DNA, 85 bp, synthetic construct; identical to SEQ ID NO: 2
  misc_feature 1..85: note = Synthetic- 2-L50 sequence for TraNS device
  modified_base 50: mod_base = OTHER; note = IANBD modification
  source 1..85: mol_type = other DNA

SEQ ID NO: 20 - DNA, 85 bp, synthetic construct; identical to SEQ ID NO: 2
  misc_feature 1..85: note = Synthetic- 2-L42 sequence for TraNS device
  modified_base 42: mod_base = OTHER; note = IANBD modification
  source 1..85: mol_type = other DNA

SEQ ID NO: 21 - DNA, 45 bp, synthetic construct
  misc_feature 1..45: note = Synthetic- 2-L45 sequence for TraNS device
  modified_base 45: mod_base = OTHER; note = IANBD modification
  source 1..45: mol_type = other DNA
cgtggaccaggcatgtacccgctctccgcgttgttattggtcacc

SEQ ID NO: 22 - DNA, 65 bp, synthetic construct; identical to SEQ ID NO: 4
  misc_feature 1..65: note = Synthetic- 3-L9 sequence for TraNS device
  modified_base 9: mod_base = OTHER; note = IANBD modification
  source 1..65: mol_type = other DNA

SEQ ID NO: 23 - DNA, 65 bp, synthetic construct; identical to SEQ ID NO: 4
  misc_feature 1..65: note = Synthetic- 3-L4 sequence for TraNS device
  modified_base 4: mod_base = OTHER; note = IANBD modification
  source 1..65: mol_type = other DNA

SEQ ID NO: 24 - DNA, 65 bp, synthetic construct; identical to SEQ ID NO: 4
  misc_feature 1..65: note = Synthetic- 3-L17 sequence for TraNS device
  modified_base 17: mod_base = OTHER; note = IANBD modification
  source 1..65: mol_type = other DNA

SEQ ID NO: 25 - DNA, 43 bp, synthetic construct
  misc_feature 1..43: note = Synthetic- aTraNS-2 sequence for TraNS device
  source 1..43: mol_type = other DNA
ctctccgcgttgttattggtcaccggttgtggagccgcggttc

SEQ ID NO: 26 - DNA, 65 bp, synthetic construct
  misc_feature 1..65: note = Synthetic- aTraNS-5 sequence for TraNS device
  source 1..65: mol_type = other DNA
gtaccttcggtgtagctcgggtgcggtttttttttttttccgctcgtggaccaggcatgtacccg

SEQ ID NO: 27 - DNA, 23 bp, synthetic construct
  misc_feature 1..23: note = Synthetic- target sequence for TraNS device
  source 1..23: mol_type = other DNA
agcggattgtgggtcttaccgca

SEQ ID NO: 28 - DNA, 22 bp, synthetic construct
  misc_feature 1..22: note = Synthetic- Target sequence for TraNS device
  source 1..22: mol_type = other DNA
tagcttatcagactgatgttga

SEQ ID NO: 29 - DNA, 21 bp, synthetic construct
  misc_feature 1..21: note = Synthetic- Target sequence for TraNS device
  source 1..21: mol_type = other DNA
atcacattgccagggattacc

SEQ ID NO: 30 - DNA, 21 bp, synthetic construct
  misc_feature 1..21: note = Synthetic- Target sequence for TraNS device
  source 1..21: mol_type = other DNA
atcacattgccagggattacc

SEQ ID NO: 31 - DNA, 23 bp, synthetic construct
  misc_feature 1..23: note = Synthetic- Hairpin sequence for TraNS device
  source 1..23: mol_type = other DNA
tgcggtaagacccacaatccgct

SEQ ID NO: 32 - DNA, 21 bp, synthetic construct
  misc_feature 1..21: note = Synthetic- Hairpin sequence for TraNS device
  source 1..21: mol_type = other DNA
tcagactcaacatcatgtctg

SEQ ID NO: 33 - DNA, 26 bp, synthetic construct
  misc_feature 1..26: note = Synthetic- Hairpin sequence for TraNS device
  source 1..26: mol_type = other DNA
tggtaatccctggcaatgtgatgatt

SEQ ID NO: 34 - DNA, 30 bp, synthetic construct
  misc_feature 1..30: note = Synthetic- Hairpin sequence for TraNS device
  source 1..30: mol_type = other DNA
tattcccctagaatcgaatctgtgggaatt

SEQ ID NO: 35 - DNA, 150 bp, synthetic construct
  misc_feature 1..150: note = Synthetic- complementary helix sequences and hairpin for Fig 3D
  source 1..150: mol_type = other DNA
cgtggaccaggcatgtacccgctctccgcgttgttattggtcaggttgtatactgcggcgggcctgcggtaacacccagaatccgctcgaccgaaaaatcgattgtgtccggttgtggagccgcggttcgtaccttcggtgtagctcggg